(19) United States
(12) Patent Application Publication
(10) Pub. No.: US 2025/0075263 A1
(43) Pub. Date: Mar. 6, 2025

(54) METHODS AND COMPOSITIONS FOR ENHANCING SIGNAL DETECTION IN SITU USING METAL NANOPARTICLES

(71) Applicant: 10x Genomics, Inc., Pleasanton, CA (US)

(72) Inventors: Zachary W. BENT, Pleasanton, CA (US); Yuwei LI, Belmont, CA (US); Mark STAPLETON, Walnut Creek, CA (US); Weiyi TANG, Dublin, CA (US); Bixun WANG, San Ramon, CA (US)

(21) Appl. No.: 18/818,462

(22) Filed: Aug. 28, 2024

Related U.S. Application Data
(60) Provisional application No. 63/535,225, filed on Aug. 29, 2023.

(51) Int. Cl. C12Q 1/6841; C12Q 1/682; G01N 21/64; G01N 33/58

(52) U.S. Cl. CPC C12Q 1/6841; C12Q 1/682; G01N 21/6428; G01N 33/582; G01N 2021/6439

(57) ABSTRACT

The present disclosure relates in some aspects to methods and compositions for in situ detection of an analyte in a biological sample embedded in a matrix that is attached to metal nanoparticles. In some embodiments, the detection involves generation of a fluorescent signal that is enhanced by the metal nanoparticles. The fluorescent signal can be detected at a 3-dimensional (3D) location in the matrix which corresponds to the 3D location of the analyte in the biological sample.

### CROSS-REFERENCE TO RELATED APPLICATION

This application claims priority to U.S. Provisional Patent Application No. 63/535,225, filed Aug. 29, 2023, entitled “METHODS AND COMPOSITIONS FOR ENHANCING SIGNAL DETECTION IN SITU USING METAL NANOPARTICLES,” which is herein incorporated by reference in its entirety for all purposes.

### FIELD

The present disclosure relates in some aspects to methods and compositions for in situ detection of analytes in a biological sample embedded in a matrix attached to metal nanoparticles.

### BACKGROUND

In situ detection and analysis methods allow one to study the subcellular distribution of gene activity (as evidenced, e.g., by expressed gene transcripts), and have the potential to provide crucial insights in the fields of developmental biology, oncology, immunology, histology, etc. In some cases, in situ detection and analysis methods rely on the generation and detection of fluorescent signals associated with analytes in the biological sample. Weak fluorescent signals associated with analytes may compromise the ability to detect, quantify, and analyze the biological sample. Provided herein are methods and compositions that address these and other needs.

### SUMMARY

In some aspects, provided herein is a method for sample analysis, comprising: providing a biological sample embedded in a hydrogel matrix that is attached to metal nanoparticles; generating a fluorescent signal associated with an analyte in the biological sample; and detecting the fluorescent signal at a 3-dimensional (3D) location in the hydrogel matrix which corresponds to the 3D location of the analyte in the biological sample.

In any of the embodiments herein the metal nanoparticles attached to the hydrogel matrix can be distributed throughout the 3D volume of the hydrogel matrix.

In any of the embodiments herein the metal nanoparticles can enhance the fluorescent signal via metal-enhanced fluorescence (MEF). In any of the embodiments herein the location of the fluorescent signal can be between about 5 nanometers (nm) and about 90 nm from one or more of the metal nanoparticles attached to the hydrogel matrix. In any of the embodiments herein the fluorescent signal can be generated from a fluorescent moiety that is between about 5 nm and about 90 nm from one or more of the metal nanoparticles attached to the hydrogel matrix.

In any of the embodiments herein the analyte can be a fluorescent analyte and the fluorescent signal can be fluorescence of the analyte. In any of the embodiments herein the analyte can be a nucleic acid analyte or a non-nucleic acid analyte. In any of the embodiments herein generating the fluorescent signal associated with the analyte can comprise contacting the biological sample with a detectably labeled probe that binds directly or indirectly to the analyte, a primary probe that binds to the analyte, or a product of the primary probe, and can use the detectably labeled probe to generate a fluorescent signal. In some embodiments, the generating the fluorescent signal associated with the analyte comprises contacting the biological sample with a detectably labeled probe that binds directly or indirectly to the analyte, optionally via a primary probe that binds to the analyte or via a product (e.g., RCA product) of the primary probe, and using the detectably labeled probe to generate a fluorescent signal. In any of the embodiments herein the detectably labeled probe can comprise a fluorescent label. In any of the embodiments herein the detectably labeled probe (a) can bind to a primary probe that directly binds to the analyte or a product of the primary probe, or (b) can bind to an intermediate probe that binds directly or indirectly to a primary probe that directly binds to the analyte or a product of the primary probe. In some embodiments, the detectably labeled probe (a) binds to a primary probe that directly binds to the analyte, (b) binds to a product of a primary probe that directly binds to the analyte, (c) binds to an intermediate probe that binds directly or indirectly to a primary probe that directly binds to the analyte, or (d) binds to an intermediate probe that binds directly or indirectly to a product of a primary probe that directly binds to the analyte. In some embodiments, the detectably labeled probe, the primary probe, and/or the intermediate probe are nucleic acid probes. In some embodiments, the analyte is an RNA (e.g., mRNA), genomic DNA, or cDNA.

In any of the embodiments herein the primary probe and the intermediate probe can each be independently selected from the group consisting of: a probe comprising a 3′ or 5′ overhang. In some embodiments the 3′ or 5′ overhang comprises one or more barcode sequences and a probe comprising a 3′ overhang and a 5′ overhang. In some embodiments the 3′ overhang and the 5′ overhang each independently comprises one or more barcode sequences; a circular probe; a circularizable probe or probe set; a probe or probe set comprising a split hybridization region configured to hybridize to a splint. In some embodiments the split hybridization region comprises one or more barcode sequences; and a combination thereof.

In any of the embodiments herein generating the fluorescent signal associated with the analyte can comprise performing rolling circle amplification (RCA) to generate an RCA product (RCP). In some embodiments the RCA is performed using as template a circular or circularized probe that is hybridized to the analyte, a product thereof, or to a probe bound directly or indirectly thereto. In some embodiments the detectably labeled probe binds directly or indirectly to the RCP. In some embodiments, the RCA is performed using as template a circular or circularized probe that is hybridized to the analyte, to a product of the analyte, or to a probe bound directly or indirectly to the analyte or product of the analyte, optionally wherein the detectably labeled probe binds directly or indirectly to the RCP. In any of the embodiments herein any one or more of the analyte, the detectably labeled probe, the intermediate probe, the primary probe, and the RCP, can be immobilized in the hydrogel matrix. In any of the embodiments herein the fluorescent signal can be a first fluorescent signal, the detectably labeled probe can be a first detectably labeled probe, and the method can further comprise: contacting the biological sample with a subsequent detectably labeled probe that binds directly or indirectly to a subsequent analyte or product thereof in the biological sample and using the subsequent detectably labeled probe to generate a subsequent fluorescent signal; and detecting the subsequent fluorescent signal at a 3D location in the hydrogel matrix which corresponds to the 3D location of the subsequent analyte in the biological sample.

In some embodiments the method comprises removing the first detectably labeled probe from the biological sample, optionally via one or more wash steps, prior to contacting the sample with the subsequent detectably labeled probe. In some embodiments the metal nanoparticles remain attached to the hydrogel matrix at least until detecting the subsequent fluorescent signal. In any of the embodiments herein the metal nanoparticles can enhance the subsequent fluorescent signal via metal-enhanced fluorescence. In some embodiments the first analyte and the subsequent analyte are the same or different.

In any of the embodiments herein the method can comprise contacting the biological sample with matrix-forming monomers and polymerizing the matrix-forming monomers to form the hydrogel matrix. In some embodiments the matrix-forming monomers comprise one or more matrix-forming monomer species. In some embodiments one or more of the matrix-forming monomer species comprises the metal nanoparticles. In any of the embodiments herein the metal nanoparticles can be provided with the matrix-forming monomers prior to and/or during forming the hydrogel matrix. In some embodiments the metal nanoparticles are not provided with the matrix-forming monomers prior to and/or during forming the hydrogel matrix.

In any of the embodiments herein the metal nanoparticles can be contacted with and/or attached to the hydrogel matrix after the hydrogel matrix is formed. In any of the embodiments herein the metal nanoparticles can be attached to the hydrogel matrix via an attachment moiety that is linked to the metal nanoparticles. In some embodiments the attachment moiety is attached to an anchoring moiety in the hydrogel matrix, wherein the attachment moiety and the anchoring moiety are a ligand-ligand binding pair, or functional moieties that can react with each other.

In any of the embodiments herein the hydrogel matrix can be covalently attached to the metal nanoparticles. In any of the embodiments herein the attachment between the metal nanoparticles and the hydrogel matrix can be achieved by a nucleophilic addition, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, or Cu(I)-catalyzed azide-alkyne cycloaddition (CuAAC) reaction. In any of the embodiments herein the attachment between the metal nanoparticles and the hydrogel matrix can be achieved by click reaction or Michael addition. In some embodiments the anchoring moiety comprises an acrylamide group, and the attachment moiety comprises a nucleophilic group capable of reacting with the acrylamide group of the anchoring moiety to form a covalent attachment. In some embodiments the nucleophilic group capable of reacting with the acrylamide group is an amine.

In some embodiments the attachment moiety has the formula (I):

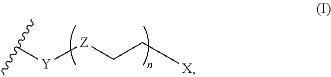

wherein Z is CH₂ or O, Y is S or O, X is —NH₂ or —OH, and n is an integer between 1 to 50, and wherein the wavy line denotes the link of the attachment moiety to the nanoparticle.

In some embodiments the attachment moiety has the formula (I-1)

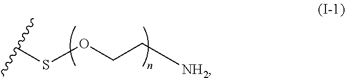

wherein n is an integer between 1-50, and wherein the wavy line denotes the link of the attachment moiety to the nanoparticle.

In any of the embodiments herein the metal nanoparticles can comprise chromium, copper, gold, iron, nickel, platinum, silver, tin, zinc, or a combination thereof. In any of the embodiments herein the metal nanoparticles can comprise gold. In some embodiments the attachment moiety is linked to the metal nanoparticles via an Au—S bond. In any of the embodiments herein the metal nanoparticles can comprise silver. In some embodiments the attachment moiety is linked to the nanoparticle via an Ag—S or Ag—O bond. In some embodiments the attachment moiety is biotin and the anchoring moiety is streptavidin, or wherein the attachment moiety is streptavidin and the anchoring moiety is biotin.

In any of the embodiments herein the average diameter of the metal nanoparticles can be between about 5 nanometers (nm) and about 250 nm. In any of the embodiments herein the concentration of metal nanoparticles attached to the hydrogel matrix can be between about 10\hat{ }6 to 10\hat{ }16 metal nanoparticles per milliliter (mL).

In any of the embodiments herein the fluorescent signal can be enhanced in comparison to a fluorescent signal generated by the same method in the absence of the metal nanoparticles. In any of the embodiments herein the intensity of the fluorescent signal can be enhanced in one or more emission spectra. In any of the embodiments herein the metal nanoparticles can comprise one or more different metal nanoparticle species. In some embodiments two or more of the different metal nanoparticle species have different average diameters and/or comprise different metals or combinations thereof. In some embodiments two or more of the different metal nanoparticle species enhance the intensity of fluorescent signals in different emission spectra.

In any of the embodiments herein the biological sample can be a cleared biological sample. In any of the embodiments herein the biological sample can be cleared before or after hydrogel matrix formation. In any of the embodiments herein the biological sample can be cleared before detecting the fluorescent signal. In any of the embodiments herein the biological sample can be cleared after the metal nanoparticles are attached to the hydrogel matrix. In some embodiments the biological sample is cleared after immobilizing the analyte or a product thereof, the detectably labeled probe, the first detectably labeled probe, the second detectably labeled probe, the primary probe, the intermediate probe, and/or the RCP in the hydrogel matrix.

In any of the embodiments herein the biological sample can be provided on a solid support. In some embodiments the biological sample is a tissue section. In some embodiments the biological sample is a cell pellet, a cell block, or a section thereof. In any of the embodiments herein the biological sample can be non-homogenized. In some embodiments the biological sample is selected from the group consisting of a formalin-fixed, paraffin-embedded (FFPE) sample, a frozen tissue sample, and a fresh tissue sample. In any of the embodiments herein the biological sample can be a section having a thickness of at least 0.2 μm, at least 1 μm, or at least 5 μm.

In some aspects, provided herein is a method for sample analysis, comprising: providing a biological sample embedded in a hydrogel matrix that is attached to metal nanoparticles; contacting the sample with a circular probe that hybridizes to an analyte or contacting the sample with a circularizable probe that hybridizes to the analyte and circularizing the circularizable probe to form a circularized probe; performing rolling circle amplification (RCA) using the circular or circularized probe as template to generate an RCA product (RCP); contacting the biological sample with a first detectably labeled probe that binds directly or indirectly to the RCP and detecting a fluorescent signal generated from the first detectably labeled probe at a 3-dimensional (3D) location in the hydrogel matrix which corresponds to the 3D location of the analyte in the biological sample; removing the first detectably labeled probe from the biological sample; and contacting the biological sample with a subsequent detectably labeled probe that binds directly or indirectly to the RCP and detecting a subsequent fluorescent signal generated from the subsequent detectably labeled probe at a 3-dimensional (3D) location in the hydrogel matrix which corresponds to the 3D location of the analyte in the biological sample. In some embodiments, the method has one or more wash steps. In some embodiments the metal nanoparticles remain attached to the hydrogel matrix at least until detecting the subsequent fluorescent signal. In some embodiments the metal nanoparticles enhance the first fluorescent signal and subsequent fluorescent signal via metal-enhanced fluorescence (MEF).

In some embodiments, the analyte is a first analyte, the fluorescent signal is a first fluorescent signal, the detectably labeled probe is a first detectably labeled probe, and fluorescent signal is at a first 3D location. In some embodiments, the method further comprises: contacting the biological sample with a second detectably labeled probe that binds directly or indirectly to a second analyte or a product of the second analyte in the biological sample; generating a second fluorescent signal associated with a second analyte in the biological sample; and detecting a second fluorescent signal at a second 3D location in the hydrogel matrix which corresponds to the 3D location of the second analyte in the biological sample. In some embodiments, the first and the second 3D locations are different. In some embodiments, the first detectably labeled probe and the second detectably labeled probe are the same or different. In some embodiments, at least some metal nanoparticles enhance the first fluorescent signal via metal-enhanced fluorescence. In some embodiments, at least some metal nanoparticles enhance the second fluorescent signal via metal-enhanced fluorescence. In some embodiments, the metal nanoparticles that enhance the first fluorescent signal are different from the metal nanoparticles that enhance the second fluorescent signal. In some embodiments, the emission spectrum of the first fluorescent signal is different from the emission spectrum of the second fluorescent signal. In some embodiments, the metal nanoparticles that enhance the first fluorescent signal comprise Au and the metal nanoparticles that enhance the second fluorescent signal comprise Ag, or vice versa.

In some embodiments, the analyte is a first analyte, the fluorescent signal is a first fluorescent signal, and the fluorescent signal is at a first 3D location. In some embodiments, the method further comprises: generating a second fluorescent signal associated with a second analyte in the biological sample, and detecting the second fluorescent signal at a second 3D location in the hydrogel matrix which corresponds to the 3D location of the second analyte in the biological sample. In some embodiments, the first and the second 3D locations are different. In some embodiments, the metal nanoparticles enhance the first fluorescent signal and/or second fluorescent signal via MEF. In some embodiments, the emission spectrum of the first fluorescent signal is different from the emission spectrum of the second fluorescent signal. In some embodiments, a first subset of the metal nanoparticles enhances the first fluorescent signal and a second subset of the metal nanoparticles enhances the second fluorescent signal. In some embodiments, the first subset of the metal nanoparticles enhances the first fluorescent signal more than the second fluorescent signal, and the second subset of the metal nanoparticles enhances the second fluorescent signal more than the first fluorescent signal. In some embodiments, the first subset of the metal nanoparticles comprises Ag. In some embodiments, the second subset of the metal nanoparticles comprises Au.

In one aspect, provided herein is a kit for analyzing a biological sample, the kit comprising: a) a probe configured to bind directly or indirectly to an analyte in the biological sample, wherein the probe comprises a fluorescent label; and b) hydrogel matrix forming monomers comprising metal nanoparticles, wherein the metal nanoparticles are capable of enhancing, via metal-enhanced fluorescence (MEF), a fluorescent signal generated from the fluorescent label.

In one aspect, provided herein is a kit for analyzing a biological sample, the kit comprising: a) a probe configured to bind directly or indirectly to a target analyte in the biological sample, wherein the probe comprises a fluorescent label; b) hydrogel matrix forming monomers; and c) metal nanoparticles configured to be attached to the hydrogel matrix forming monomers and/or to a hydrogel matrix formed from the hydrogel matrix forming monomers, wherein the metal nanoparticles are capable of enhancing, via metal-enhanced fluorescence (MEF), a fluorescent signal generated from the fluorescent label.

In one aspect, provided herein is a system for analyzing a biological sample, the system comprising: a) a probe configured to bind directly or indirectly to an analyte in the biological sample, wherein the probe comprises a fluorescent label; and b) hydrogel matrix forming monomers comprising metal nanoparticles, wherein the metal nanoparticles are capable of enhancing, via metal-enhanced fluorescence (MEF), a fluorescent signal generated from the fluorescent label.

In one aspect, provided herein is a system for analyzing a biological sample, the system comprising: a) a probe configured to bind directly or indirectly to a target analyte in the biological sample, wherein the probe comprises a fluorescent label; b) hydrogel matrix forming monomers; and c) metal nanoparticles configured to be attached to the hydrogel matrix forming monomers and/or to a hydrogel matrix formed from the hydrogel matrix forming monomers, wherein the metal nanoparticles are capable of enhancing, via metal-enhanced fluorescence (MEF), a fluorescent signal generated from the fluorescent label.

### BRIEF DESCRIPTION OF THE DRAWINGS

The drawings illustrate certain features and advantages of this disclosure. These embodiments are not intended to limit the scope of the appended claims in any manner.

FIGS. 1A-1B depict detection of fluorescent signals in a matrix embedding a biological sample (shown as wavy line matrix), without (FIG. 1A) or with (FIG. 1B) metal nanoparticles (shown as blank circles) attached to the matrix. Fluorescent signals, shown as stars, are enhanced in the presence of metal nanoparticles, for example by metal-enhanced fluorescence (MEF).

FIG. 2 is a schematic illustrating fluorescent signals (shown as stars) and metal nanoparticles of more than one species attached to a hydrogel matrix embedding a biological sample (shown as a matrix with wavy lines). In FIG. 2, one metal nanoparticle species is shown as solid circles while another metal nanoparticle species is shown as blank circles. Fluorescent signal 1 is shown as solid stars, while florescent signal 2 is shown as blank stars.

FIG. 3 shows a schematic illustrating an exemplary method for providing a hydrogel matrix attached to metal nanoparticles.

FIG. 4 shows images of fluorescent signals detected in situ in a biological sample mounted on glass slides or gold coated slides, as described in Example 1.

FIG. 5 shows quantification of the number of individual fluorescent signals (features) detected per nuclei area in cortex or dentate gyrus of samples on glass or gold-coated slides, in channels corresponding to indicated transcripts or negative control, as described in Example 1.

FIG. 6 illustrates a workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ detection of target analytes such as nucleic acids in biological samples using microscopic imaging can provide valuable information regarding analyte abundance and localization in situ. Thus, in situ assays are important tools, for example, for understanding the molecular basis of cell identity and developing treatment for diseases. In some cases, in situ detection and analysis methods rely on the generation and detection of fluorescent signals associated with analytes in the biological sample. Weak fluorescent signals associated with analytes may compromise the ability to detect, quantify, and analyze the biological sample.

A metallic surface in proximity to a fluorophore can have effects on optical properties associated with the fluorophore that may be beneficial for detection, such as enhancing a fluorescent signal generated from the fluorophore. This enhancement of a fluorescent signal by a proximal metal is generally known as metal-enhanced fluorescence (MEF), and may occur by a variety of mechanisms, for example as described in Jeong et al., Biosens. Bioelectron. 111:102-116, 2018, which is herein incorporated by reference in its entirety. For example, MEF can increase quantum yield and improve photostability. In some instances, MEF can result in increased emission intensity, leading to improved signal-to-noise ratios, as a result of surface plasmon resonance (SPR).

Hydrogel tissue chemistry can be employed in in situ assays for a variety of applications. For example, embedding a biological sample in a hydrogel can allow for the preservation of certain components in the sample (e.g., analytes such as nucleic acids) while removing other components (e.g., lipids and proteins that increase autofluorescence, or probes that are bound non-specifically), all while maintaining the spatial fidelity of analytes of interest in the sample. However, detection of analytes in a hydrogel in some instances may be hampered by an insufficiently strong fluorescent signal for optimal detection and downstream analysis.

In some aspects, provided herein are methods and compositions for generating fluorescent signals in situ in a biological sample embedded in a hydrogel, wherein the hydrogel comprises a matrix that is attached to metal nanoparticles. In some aspects, the method comprises generating a fluorescent signal associated with an analyte in the biological sample. In some embodiments, the method comprises detecting the fluorescent signal at a 3-dimensional (3D) location in the matrix, which corresponds to the 3D location of the analyte in the biological sample. In some embodiments, the method comprises generating a first fluorescent signal associated with an analyte in the biological sample and detecting a first fluorescent signal at a first 3-dimensional (3D) location in the matrix, and generating a second fluorescent signal associated with an analyte in the biological sample and detecting a second fluorescent signal at a second 3-dimensional (3D) location in the matrix. In some aspects, the metal nanoparticles attached to the matrix enhance the fluorescent signal, for example via metal-enhanced fluorescence (MEF). The nanoparticles can be attached to the matrix in any suitable manner, such as via a covalent or non-covalent attachment. For example, the nanoparticles (such as gold nanoparticles, or metal nanoparticles comprising any suitable metal) can be chemically conjugated to the matrix.

The methods and compositions provided herein present several advantages. In some aspects, the methods provided herein can substantially improve signal intensity (e.g., by at least 10-fold) of fluorescent signals corresponding to analytes provided in situ (e.g. rolling circle products (RCPs) produced from rolling circle amplification (RCA) reactions). The methods and compositions provide high sensitivity and easy adaptability, presenting new opportunities for a wide variety of in situ assays employing hydrogel chemistry.

In some aspects, providing metal nanoparticles attached to a matrix is advantageous in comparison to other methods that may use MEF to enhance fluorescent signals. For example, gold-coated slides could theoretically enhance fluorescent signals in a biological sample mounted on the slide, but would not allow for enhancement of signals throughout a three dimensional sample due to the requirement for close proximity to the metal, as in the provided methods. The attachment of nanoparticles to the matrix is also advantageous because it can facilitate, for example, multiple detection steps, which can be separated by one or more washing and/or other processing steps, while retaining the same metal nanoparticles in the hydrogel in the multiple steps. Without attachment to the hydrogel, new metal nanoparticles would need to be provided in the multiple detection steps, greatly increasing the cost and complexity of the procedure.

In some aspects, provided herein is a method for sample analysis, comprising providing a biological sample embedded in a hydrogel matrix that is attached to metal nanoparticles. In some embodiments, the method comprises generating a fluorescent signal associated with an analyte in the biological sample. In some embodiments, the method comprises detecting the fluorescent signal at a 3-dimensional (3D) location in the hydrogel matrix which corresponds to the 3D location of the analyte in the biological sample.

### II. Metal Nanoparticles and Related Methods and Compositions

In some aspects, provided herein are metal nanoparticles and related methods and compositions. In some aspects, the metal nanoparticles are attached to a matrix embedding a biological sample, such as the matrix of a hydrogel (i.e., a hydrogel matrix). In some aspects, the metal nanoparticles are distributed throughout the 3D volume of the matrix. In some aspects, the metal nanoparticles enhance a fluorescent signal associated with an analyte in the biological sample. In some aspects, the metal nanoparticles enhance the fluorescent signal via metal-enhanced fluorescence (MEF).

In some aspects, provided herein is a method for sample analysis, comprising: providing a biological sample embedded in a hydrogel comprising a matrix that is attached to metal nanoparticles; generating a fluorescent signal associated with an analyte in the biological sample; and detecting the fluorescent signal at a 3-dimensional (3D) location in the matrix which corresponds to the 3D location of the analyte in the biological sample.

### A. Metal Nanoparticles

In some aspects, provided herein are metal nanoparticles. In some aspects, the metal nanoparticles are attached to the matrix of a hydrogel embedding a biological sample. In some aspects, the metal nanoparticles enhance a fluorescent signal generated in situ in the biological sample embedded in the hydrogel, for example via metal-enhanced fluorescence (MEF).

The metal nanoparticles can comprise any suitable metal for mediating metal-enhanced fluorescence (MEF), including chromium (Cr), copper (Cu), gold (Au), iron (Fe), nickel (Ni), palladium (Pd), platinum (Pt), silver (Ag), tin (Sn), zinc (Zn), any alloys thereof, or any combination thereof. In some embodiments, the metal nanoparticles comprise a single metal (e.g., Au). In some embodiments, the metal nanoparticles comprise more than one type of metal (e.g., Ag and Au).

In some embodiments, the metal of the metal nanoparticles is Au. In some embodiments, the metal of the metal nanoparticles is Ag. In some embodiments, the metal of the metal nanoparticles is Cu. In some embodiments, the metal of the metal nanoparticles is Zn. In some embodiments, the metal of the metal nanoparticles is Cr. In some embodiments, the metal of the metal nanoparticles is Ni. In some embodiments, the metal of the metal nanoparticles is Sn. In some embodiments, the metal of the metal nanoparticles is Fe. In some embodiments, the metal of the metal nanoparticles is Pd. In some embodiments, the metal of the metal nanoparticles is Pt. In some embodiments, the surface of the metal nanoparticles may be at least partially oxidized, and it should be understood that the partially oxidized metal nanoparticles are also encompassed by the present invention. In some embodiments, no more than about 50%, such as no more than about any of 40%, 30%, 20%, 10%, 5%, 2%, 1%, or 0.1% of the surface of the metal nanoparticles are oxidized. In some embodiments, at least about 50%, 60%, 70%, 80%, 90%, 95%, 98%, or 99% of the surface of the metal nanoparticles are oxidized.

In some embodiments, the metal nanoparticles attached to the matrix are distributed throughout the 3D volume of the hydrogel. In some aspects, the distribution of the metal nanoparticles throughout the 3D volume of the hydrogel is substantially uniform. In some aspects, the distribution of the metal nanoparticles throughout the 3D volume of the hydrogel allows for fluorescent signals throughout the 3D volume to be enhanced. Metal-enhanced fluorescence (MEF) typically occurs from the interaction of a fluorophore and a metal surface separated by a distance of about 5-90 nanometers (nm). Thus, in some aspects, the location of one or more detected fluorescent signals is between about 5 nm and about 90 nm from one or more of the metal nanoparticles attached to the matrix. In some embodiments, the fluorescent signal is generated from a fluorescent moiety (e.g. a fluorophore) that is between about 5 nm and about 90 nm from one or more of the metal nanoparticles attached to the matrix. The metal nanoparticles attached to the matrix can be provided at any suitable concentration. For example, the concentration can be adjusted to optimize the average distance between fluorophores and metal nanoparticles for MEF to occur. In some embodiments, the concentration of metal nanoparticles attached to the matrix is between about 10\hat{ }7 and about 10\hat{ }16 metal nanoparticles per milliliter (mL). In some embodiments, the concentration of metal nanoparticles attached to the matrix is between about 10\hat{ }6 and about 10\hat{ }15 metal nanoparticles per milliliter (mL). In some embodiments, the concentration of metal nanoparticles attached to the matrix is between about 10\hat{ }7 and about 10\hat{ }15 metal nanoparticles per milliliter (mL). In some embodiments, the concentration of metal nanoparticles attached to the matrix is about 10\hat{ }6, about 10\hat{ }7, about 10\hat{ }8, about 10\hat{ }9, about 10\hat{ }10, about 10\hat{ }11, about 10\hat{ }12, about 10\hat{ }13, about 10\hat{ }14, about 10\hat{ }15, or about 10\hat{ }16 metal nanoparticles per mL.

In some embodiments, the average diameter of the metal nanoparticles is between about 5 nm and about 250 nm, such as about any of 50 nm to 200 nm, 50 nm to 150 nm, or 50 nm to 100 nm. In some embodiments, the average diameter of the metal nanoparticles is between about 5 nm and about 250 nm, such as about any of 50 nm to 200 nm, 50 nm to 150 nm, or 50 nm to 100 nm.

In some embodiments, the metal nanoparticles comprise one or more different metal nanoparticle species, which may have different properties. In some embodiments, the metal nanoparticles comprise one or more different nanoparticle species that are selected based on one or more fluorescent signals to be detected and/or enhanced in the biological sample. For example, different metal nanoparticle species can comprise different metals (e.g. a first species comprising gold nanoparticles and a second species comprising silver nanoparticles), and/or different sizes (e.g. a first species having an average diameter of 50 nm and a second species having an average diameter of 100 nm). Different metal nanoparticle species may be provided at different concentrations in the matrix. In some embodiments, the different metal nanoparticle species can enhance the intensity of fluorescent signals in different emission spectra.

The optical and electronic properties of metal nanoparticles are tunable by changing the metal type, size, shape, surface chemistry, or aggregation state. For example, the size of metal nanoparticles can be tuned to quench certain fluorophore wavelengths while enhancing others. In certain embodiments, the geometry and size of the nanoparticles determine the properties of the localized surface plasmons they support. In some embodiments, at the plasmon resonance wavelength of a metal nanoparticle, the light intensity in the near field of the nanoparticle (also known as the fringing field) is enhanced strongly relative to the incident optical wave.

### B. Hydrogel Matrix Formation and Attachment of Metal Nanoparticles

In some embodiments, the method comprises embedding the biological sample in a hydrogel matrix. In some aspects, a hydrogel comprises a matrix component (e.g. a porous, permeable solid) and a fluid component (e.g. interstitial fluid comprising water). In some embodiments, the method comprises contacting the biological sample with matrix-forming monomers and polymerizing the matrix-forming monomers to form the matrix. In some embodiments, the metal nanoparticles are attached to the matrix. In some embodiments, the metal nanoparticles are distributed throughout the 3D volume of the matrix.

In some embodiments, the metal nanoparticles are attached to the matrix of the hydrogel embedding the biological sample. The metal nanoparticles can be attached to the hydrogel embedding the biological sample by any suitable method, and the attachment can be covalent or non-covalent. In some aspects, it will be apparent to one skilled in the art that any other suitable component in addition to the metal nanoparticles (e.g. probes or analytes) can be attached to (e.g. immobilized in) the matrix in accordance with the methods described herein. In some embodiments, in addition to metal nanoparticles attached to the matrix of the hydrogel embedding the biological sample, metal nanoparticles can also be provided to be non-covalently incorporated or trapped in the matrix of the hydrogel. In some cases, the pore size of the hydrogel can be adjusted to trap or incorporate a suitable amount of metal nanoparticles.

In some embodiments, the metal nanoparticle is linked to an attachment moiety that can be attached to the hydrogel matrix. In some embodiments, the attachment moiety is attached to an anchoring moiety in the matrix embedding the biological sample. The attachment can be covalent (e.g., crosslinking) or non-covalent (e.g., interaction between a ligand-ligand binding pair). In some embodiments, the attachment moiety is any suitable cross-linkable moiety.

In some embodiments, the metal nanoparticle is linked to a labelling agent comprising an attachment moiety that can be attached to the hydrogel matrix. In some embodiments, one or more labeling agents capable of binding to or otherwise coupling to one or more features may be used to provide the metal nanoparticle. For example, each labelling agent can comprise a binding moiety capable of binding to an a biological moiety (e.g., an endogenous cell feature) in the biological sample, (e.g., a macromolecular constituent), a metal nanoparticle and an attachment moiety for attaching to the matrix. In some instances, cell features include cell surface features. In some instances, cell features include an internally localized protein. The biological moiety may include, but is not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular molecules, such as proteins, protein modifications, nuclear proteins, nuclear membrane proteins, or any combination thereof. A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof.

In some embodiments, the attachment moiety comprises a reactive group. Exemplary reactive groups for attachment to a biological sample or matrix include, but are not limited to, an amine, a thiol, an azide, an alkyne, a nitrone, an alkene, a tetrazine, a tetrazole, an acrydite or other click reactive group. In some embodiments, the attachment moiety is an acrydite moiety. In some embodiments, the acrydite is a C6 methacrylate. In some embodiments, the attachment moiety is a methacrylate C6 phosphoramidite.

In some embodiments, the attachment moiety comprises or is an electrophilic group that is capable of interacting with a reactive nucleophilic group present in the biological or matrix to provide a covalent bond. In some embodiments, the nucleophilic group can be selected from a sulfhydryl, hydroxyl and amino functional group. In some embodiments, the attachment moiety comprises or is a maleimide, haloacetamide, or NHS ester.

In some embodiments, the attachment moiety comprises or is a nucleophilic group that is capable of interacting with a reactive electrophilic group present in the biological or matrix to provide a covalent bond. In some embodiments, the attachment moiety comprises or is a thiol, phenol, amino, hydrazide, hydroxylamine, hydrazine, thiosemicarbazone, hydrazine carboxylate, or arylhydrazide.

In some embodiments, the attachment moiety comprises or is a click functional group. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the attachment moiety can comprise or be any functional group involved in click reactions. In some embodiments, such click reactions may involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the attachment moiety comprises or is a click functional group, the molecule present in the matrix to which it is capable of forming a covalent bond with comprises the complementary click functional group to that of the attachment moiety. For example, in some embodiments, the attachment moiety comprises or is an azide moiety and the molecule present in the biological sample or matrix comprises a complementary alkyne moiety, or vice versa.

In some embodiments, the attachment moiety can react with a cross-linker. In some embodiments, the attachment moiety can be part of a ligand-ligand binding pair. An exemplary attachment moiety includes an amine, amine reactive groups, acrydite, an acrydite modified entity, alkyne, biotin, azide, thiol, and a thiol-modified entity and entities suitable for click chemistry techniques. Biotin, or a derivative thereof, may be used as an anchoring moiety when the metal nanoparticle is linked to an avidin/streptavidin derivative or an anti-biotin antibody. Similarly, biotin, or a derivative thereof, may be used as an attachment moiety when the matrix includes an avidin/streptavidin derivative or an anti-biotin antibody. In some embodiments, a polyacrylamide gel matrix is co-polymerized with acrydite-modified streptavidin. In some instances, biotin or an avidin/streptavidin derivatives are attached to the matrix after the matrix is formed. In one example, a polyacrylamide gel matrix is co-polymerized with acrydite-modified streptavidin monomers and biotinylated metal nanoparticles, using a suitable acrylamide:bis-acrylamide ratio to control the cross-linking density. Digoxigenin may be used as a matrix attachment moiety and subsequently bound by an anti-digoxigenin antibody attached to the matrix. In some embodiments, a Dibenzocyclooctyne (DBCO)-azide attachment moiety can be used for matrix attachment. In some embodiments, the DBCO is reacted with the azide in a strain promoted alkyne-azide cycloaddition (SPAAC). In some embodiments, the metal nanoparticle is attached to the matrix using acrydite, NHS ester, DBCO, sulfhydryl, amine. In general, any member of a conjugate pair or reactive pair may be used as an attachment moiety to attach the metal nanoparticles to a matrix.

Attachment moieties for attachment to a matrix can include chemical cross-linking agents. Cross-linking agents typically contain at least two reactive groups that are reactive towards numerous groups, including, but not limited to, sulfhydryls and amines, and create chemical covalent bonds between two or more molecules. Attachment moieties can include cross-linking agents such as primary amines, carboxyls, sulfhydryls, carbohydrates and carboxylic acids. Cross-linking agents are commercially available (Thermo Scientific (Rockford, IL)). Suitable exemplary cross-linker reactive groups include imidoester (DMP), succinimide ester (NHS), maleimide (Sulfo-SMCC), carbodiimide (DCC, EDC) and phenyl azide. Cross-linkers may include a spacer moiety. Such spacer moieties may be functionalized. Such spacer moieties may be chemically stable. Suitable exemplary spacer moieties include polyethylene glycol, carbon spacers, cleavable (e.g., photo-cleavable or chemically cleavable) spacers and other spacers and the like.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

Matrix forming materials (e.g. matrix-forming monomers) include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions.

In some embodiments, a matrix-forming material can be introduced into a cell, for example according to the following procedure. The cells are fixed with formaldehyde and then immersed in ethanol to disrupt the lipid membrane. The matrix forming reagents are added to the sample and are allowed to permeate throughout the cell. A polymerization inducing catalyst, UV or functional cross-linkers are then added to allow the formation of a gel matrix. The un-incorporated material is washed out and any remaining functionally reactive group is quenched. Exemplary cells include any cell, human or otherwise, including diseased cells or healthy cells. Certain cells include human cells, non-human cells, human stem cells, mouse stem cells, primary cell lines, immortalized cell lines, primary and immortalized fibroblasts, HeLa cells and neurons.

In some embodiments, a matrix-forming material can be used to encapsulate a biological sample, such as a tissue sample, for example as follows. Formalin-fixed embedded tissues on glass slides are incubated with xylene and washed using ethanol to remove the embedding wax. They are then treated with Proteinase K to permeabilized the tissue. A polymerization inducing catalyst, UV or functional cross-linkers are then added to allow the formation of a gel matrix. The un-incorporated material is washed out and any remaining functionally reactive group is quenched.

In some embodiments, the matrix-forming material forms a three dimensional matrix while maintaining the spatial relationship of components of interest in the biological sample (e.g. analytes). In some embodiments, the metal nanoparticles are immobilized within the matrix by covalent attachment or through ligand-ligand interaction to the matrix.

In some embodiments, the matrix is sufficiently optically transparent or otherwise has optical properties suitable for deep three dimensional imaging for high throughput information readout, such as for detection of the RCA product using labeled probes (e.g., fluorescently labeled probes).

In some embodiments, the matrix is porous thereby allowing the introduction of reagents (e.g., primary probes, intermediate probes, and/or detectably labeled probes) into the matrix at the site of a target nucleic acid molecule immobilized in the matrix. Additional control over the molecular sieve size and density can be achieved by adding additional cross-linkers such as functionalized polyethylene glycols. In some embodiments, the target nucleic acid molecules are readily accessed by probes, enzymes, and other reagents with rapid kinetics. Porosity can result from polymerization and/or crosslinking of molecules used to make the matrix material. In some aspects, the diffusion property within the gel matrix is largely a function of the pore size. The molecular sieve size can be chosen to allow for rapid diffusion of enzymes, oligonucleotides, formamide, and other buffers used for amplification and detection (>50-nm). The molecular sieve size can also be chosen so that large DNA or RNA amplicons do not readily diffuse within the matrix (<500-nm). The porosity can be controlled by changing the cross-linking density, the chain lengths, and/or the percentage of co-polymerized branching monomers. In some embodiments, the cross-linking density, the chain lengths, and/or the percentage of co-polymerized branching monomers can be tuned to trap metal nanoparticles in addition to the metal nanoparticles attached to a matrix.

In some embodiments, the biological sample is immobilized in the hydrogel via cross-linking of the polymer material that forms the hydrogel. Cross-linking can be performed chemically and/or photochemically, or alternatively by any other hydrogel-formation.

The composition and application of the hydrogel matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

In some embodiments, a matrix can be formed using photopolymerization. In some aspects, photopolymerization involves the use of photons to initiate a polymerization reaction. The photopolymerization reaction can be initiated by a single-photon or a multiphoton excitation system as described elsewhere herein. Light can be manipulated to form specific 2D or 3D patterns and be used to initiate the photopolymerization reaction. In some embodiments, a particular shape or pattern for the 3D matrix is constructed, for example such that the matrix is generated in one part of the cell or cell derivative but not generated in another part of the cell or cell derivative. Light and patterns of light can be generated by spatial light modulators, such as a digital spatial light modulator. The spatial light modulators can employ a transmissive liquid crystal, reflective liquid crystal on silicon (LCOS), digital light processing, a digital micromirror device (DMD), or a combination thereof.

The fixative/hydrogel composition can comprise any hydrogel subunits, such as, but not limited to, poly(ethylene glycol) and derivatives thereof (e.g., PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. Agents such as hydrophilic nanoparticles, e.g., poly-lactic acid (PLA), poly-glycolic acid (PLG), poly(lactic-co-glycolic acid) (PLGA), polystyrene, poly(dimethylsiloxane) (PDMS), etc. can be used to improve the permeability of the hydrogel while maintaining patternability. Materials such as block copolymers of PEG, degradable PEO, poly (lactic acid) (PLA), and other similar materials can be used to add specific properties to the hydrogel. Crosslinkers (e.g., bis-acrylamide, diazirine, etc.) and initiators (e.g., azobisisobutyronitrile (AIBN), riboflavin, L-arginine, etc.) can be included to promote covalent bonding between interacting macromolecules in later polymerization.

Examples of functional moieties include electrophiles or nucleophiles that can form a covalent linkage by reaction with a corresponding nucleophile or electrophile, respectively, on the substrate of interest. Non-limiting examples of suitable electrophilic reactive groups can include, for example, esters including activated esters (such as, for example, succinimidyl esters), amides, acrylamides, acridines, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups can include, for example, amines, anilines, thiols, alcohols, phenols, hyrazines, hydroxylamines, carboxylic acids, glycols, heterocycles, and the like. Further non-limiting examples of functional moieties include acrydite, biotin, alkyne, and amine groups.

In some embodiments, after attachment of the metal nanoparticles (and any other suitable components, such as probes or analytes) to the matrix, the matrix can be partially or substantially cleared of certain species or classes of biomolecules, such as lipids and proteins, as by use of detergent and/or protease reagents. According to some aspects of the present disclosure, the sample can be cleared using a detergent solution, such as Triton-X or SDS. The detergent can interact with the molecules allowing the molecules to be washed out or removed. Other non-limiting examples of detergents include Triton X-100, Triton X-114, Tween-20, Tween 80, saponin, CHAPS, and NP-40. In some embodiments, the sample can be cleared using a protease reaction, such as Proteinase K. The protease can cleave or digest proteins such that the fragments or amino acids can be removed. In some embodiments, the extracellular matrix can be substantially cleared using one or more specific or nonspecific proteases. Other non-limiting examples of protease include trypsin, chemotrypsin, papain, thrombin, and pepsin.

In some embodiments, the biological sample or the matrix is immobilized onto a solid substrate, such as glass or plastic, facilitating handling and reagent exchange. In some embodiments, a matrix can be affixed to a glass slide via oxysilane-functionalization with acrylamide- or free-radical-polymerizing groups, such as methacryloxypropyltrimethoxysilane. The 3D matrix can be free-floating or otherwise not attached to a solid substrate.

Additional methods and aspects of hydrogel embedding of biological samples are described for example in Chen et al., Science 347 (6221): 543-548, 2015, the content of which is herein incorporated by reference in its entirety.

In some embodiments, the matrix-forming monomers can comprise one or more matrix-forming monomer species (e.g., different types of matrix-forming monomers). In some embodiments, the matrix-forming monomers do not themselves comprise metal nanoparticles. In some embodiments, the matrix is formed independently of the metal nanoparticles. In some embodiments, the metal nanoparticles do not substantially alter or participate in the polymerization of matrix-forming monomers to form the matrix. In some embodiments, the matrix-forming monomers can be polymerized to form the matrix in the absence of the metal nanoparticles. In some embodiments, the matrix-forming monomers can be polymerized in the presence of the nanoparticles without necessarily becoming attached to the metal nanoparticles during the polymerization. Thus, in some embodiments, the formation of the matrix and the attachment of metal nanoparticles can be performed separately.

In some embodiments, one or more of the matrix-forming monomer species can comprise the metal nanoparticles. For example, in some embodiments, the metal nanoparticles can be functionalized (e.g. linked to functional moieties) and used to cross-link the matrix-forming monomers to form the matrix. In such an example, formation of the matrix (e.g., polymerization of the matrix-forming monomers) occurs simultaneously with attachment of the metal nanoparticles to the matrix. In some embodiments, the metal nanoparticles can facilitate the polymerization of matrix-forming monomers to form the matrix. For example, in some embodiments, the metal nanoparticles (e.g., Au nanoparticles) may serve as a cross linking agent for the polymerization. In some embodiments, the metal nanoparticles remain substantially uncorroded during the polymerization.

In some embodiments, the metal nanoparticles are provided with the matrix-forming monomers prior to and/or during forming the matrix. For example, in some embodiments, the matrix-forming monomers and metal nanoparticles can be mixed together prior to being contacted with the biological sample. In some embodiments, the metal nanoparticles and metal nanoparticles can both be contacted with the biological sample prior to forming the matrix.

In some embodiments, the metal nanoparticles are not provided with the matrix-forming monomers prior to and/or during forming the matrix. In some embodiments, the metal nanoparticles are provided separately from the matrix-forming monomers. For example, in some embodiments, the matrix is formed in the absence of the metal nanoparticles, and the metal nanoparticles are subsequently contacted with (and attached to) the matrix after the matrix is formed. In some embodiments, the metal nanoparticles are contacted with and/or attached to the matrix after the matrix is formed. For example, the metal nanoparticles can be infused into the matrix after the matrix is formed. In some embodiments, the matrix is configured to allow the metal nanoparticles to be infused into the matrix without significantly altering the physical shape and stability of the matrix.

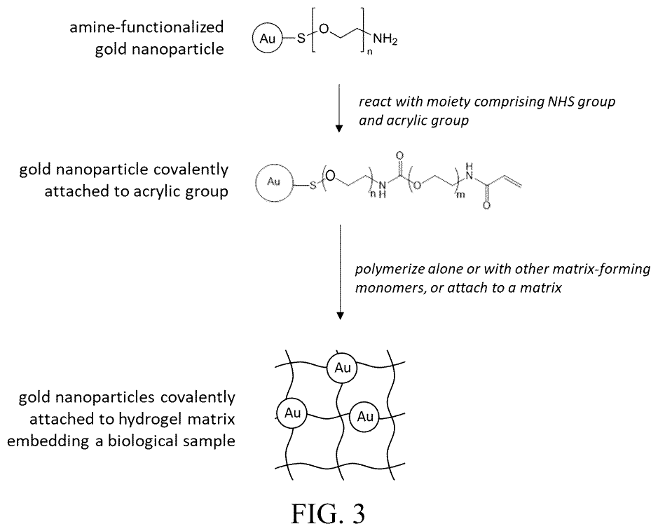

FIG. 3 shows a schematic illustrating an exemplary method for providing a hydrogel matrix attached to metal nanoparticles. Amine-functionalized gold nanoparticles are reacted with a moiety comprising an N-hydroxysuccinimide (NHS) group and acrylic group, thereby generating gold nanoparticles covalently attached to acrylic groups. In some embodiments, amine-functionalized gold nanoparticles are reacted with a moiety comprising an NHS ester moiety and acrylic group. The gold nanoparticles covalently attached to acrylic groups can then be polymerized, optionally with one or more other matrix-forming monomers to form a hydrogel matrix, or attached to a pre-existing hydrogel matrix embedding a biological sample. Any of a variety of metal nanoparticles can be attached to a matrix embedding a biological sample, as will be apparent to one of skill in the art. Any suitable functionalized metal nanoparticles can be used in conjunction with the methods herein, including custom-made or commercially available metal nanoparticles. For example, a variety of functionalized metal nanoparticles (e.g. gold nanoparticles) are commercially available in a variety of sizes, metals, and with a variety of functional groups, for example from Cytodiagnostics, such as at cytodiagnostics.com/pages/gold-nanoparticles, or from Sigma-Aldrich, such as at sigmaaldrich.com/US/en/product/aldrich/765279 and sigmaaldrich.com/US/en/substance/goldnanoparticles 1234598765.

In some embodiments, the metal nanoparticles are distributed throughout the 3D volume of the matrix (e.g., hydrogel). In some embodiments, the concentration of metal nanoparticles in the matrix is at least at or about 10⁷ metal nanoparticles per milliliter (mL), such as at least at or about any of 10⁶, 10⁷, 10⁸, 10⁹, 10¹⁰, 10¹¹, 10¹², 10¹³, 10¹⁴, 10¹⁵, or 10¹⁶ metal nanoparticles per mL of the matrix (e.g., hydrogel). In some embodiments, the concentration of metal nanoparticles in the matrix is no more than at or about 10¹⁵ metal nanoparticles per mL, such as no more than at or about any of 10¹⁶, 10¹⁵, 10¹⁴, 10¹³, 10¹², 10¹¹, 10¹⁰, 10⁹, 10⁸, or 10⁷ metal nanoparticles per mL of the matrix (e.g., hydrogel). In some embodiments, the concentration of metal nanoparticles in the matrix is between about 10⁷ to 10¹⁵ metal nanoparticles per mL of the matrix (e.g., hydrogel). In some embodiments, the metal nanoparticles are distributed substantially uniformly throughout the matrix (e.g., hydrogel). In some embodiments, the concentration of the metal nanoparticles in the matrix may not be perfectly uniform. For example, the concentration of metal nanoparticles may in some embodiments be higher at the surface than the interior of the matrix (e.g., hydrogel). For example, in methods involving infusion of nanoparticles into a pre-formed matrix, perfectly uniform distribution may be difficult to achieve. In some embodiments, the concentration of metal nanoparticles in the matrix can be tuned based on the size and species of the nanoparticles. In some cases, the species and concentration of metal nanoparticles in the matrix is selected and tuned based on the fluorescent signal to be detected (e.g., of the detectably labeled probe(s)).

In some aspects, the methods provided herein can include attaching the metal nanoparticles to the matrix. In some embodiments, the attachment is achieved through the use of an attachment moiety that is linked to the metal nanoparticles. In some embodiments, the attachment moiety mediates the attachment between the metal nanoparticles and the matrix. In some embodiments, the metal nanoparticles are attached covalently to the matrix. In some embodiments, the metal nanoparticles are attached non-covalently to the matrix. In some embodiments, the metal nanoparticles are attached to the matrix via an attachment moiety that is linked to the metal nanoparticles. In some embodiments, the attachment moiety is attached to an anchoring moiety in the matrix. In some embodiments, the attachment moiety and the anchoring moiety are a ligand-ligand binding pair. In some embodiments, the attachment moiety and the anchoring moiety are functional moieties that can react with each other.

In some embodiments, the metal nanoparticles can be attached to the matrix at any suitable step of the method. For example, the metal nanoparticles can be attached to the matrix during formation of the matrix (e.g. as described above for methods involving using matrix-forming monomers that comprise the metal nanoparticles). In some embodiments, the metal nanoparticles are present with the matrix-forming monomers prior to formation of the matrix and are attached to the matrix during matrix formation. In other embodiments, the metal nanoparticles are attached to the matrix after the matrix has been formed. Attachment after the matrix has been formed can be achieved, for example, by infusion of metal nanoparticles into the hydrogel and attachment of the metal nanoparticles to the matrix. In other embodiments, the metal nanoparticles may be present with the matrix-forming monomers prior to formation of the matrix but not attached to the matrix during matrix formation, and the metal nanoparticles can be subsequently attached to the matrix in a separate step.

In some embodiments, the metal nanoparticles are attached to the matrix via an attachment moiety. In some embodiments, the attachment moiety is linked to the metal nanoparticles before contacting the metal nanoparticles with the matrix-forming monomers or the matrix. In some embodiments, the attachment moiety is linked to the metal nanoparticles before contacting the metal nanoparticles with the matrix-forming monomers. In some embodiments, the attachment moiety is linked to the metal nanoparticles before contacting the metal nanoparticles with the matrix.

In some embodiments, the metal nanoparticles can be linked to one or more attachment moieties. In some embodiments, the attachment moieties may be the same or different. In some embodiments, each attachment moiety is independently selected from any suitable attachment moiety.

In some embodiments, the attachment moiety can be any suitable linker that forms a stable bond with metal (such as a covalent or ionic bond) and that is capable of forming a covalent or non-covalent bond with the matrix-forming monomers and/or the matrix. In some embodiments, the attachment moiety comprises a functional group capable of being linked to the metal nanoparticles and a functional group capable of being attached to the matrix. In some embodiments, the attachment moiety can be multifunctional, comprising at least two (e.g., at least any of 2, 3, 4, 5, 6, 7, or 8) different functional groups, wherein at least one functional group is capable of being linked to the metal nanoparticles and at least one functional group is capable of covalently or non-covalently attaching to the matrix (e.g. via the anchoring moiety). In some embodiments, the attachment moiety is a bifunctional moiety. In other embodiments, the attachment moiety is a multifunctional moiety. In some embodiments wherein the attachment moiety is a multifunctional moiety, and the attachment moiety comprises at least 3 (e.g., at least any of 3, 4, 5, 6, 7, or 8) functional groups.

As provided herein, the attachment moiety can be linked to the metal nanoparticles via any suitable interaction. In some embodiments, the attachment moiety is linked to the metal nanoparticles by a covalent bond. In an exemplary embodiment, the metal nanoparticles comprise Au (e.g. the metal nanoparticles are gold nanoparticles), the attachment moiety comprises S, and the attachment moiety is linked to the metal nanoparticles via an Au—S bond. In another embodiment, the metal nanoparticles comprise Ag and the attachment moiety comprises S or O, and the attachment moiety is linked to the metal nanoparticles via an Ag—S bond or Ag—O bond, respectively. In some embodiments, the metal nanoparticles comprise Cr, Fe, Cu, Zn, Sn, Ni, or any alloy thereof, or any combination thereof, and the attachment moiety comprises S or O, and the attachment moiety is linked to the metal nanoparticles via a metal-S or metal-O bond, respectively.

In some aspects, the attachment moiety is capable of forming a covalent or non-covalent bond with the matrix-forming monomer or the matrix. In some embodiments, the attachment moiety can bind with the matrix-forming monomer or the matrix via any functional group that interacts with the matrix-forming monomer or the matrix. In some embodiments, the attachment moiety can comprise a group capable of reacting with, covalently binding, or non-covalently binding to a complementary reactive group on the matrix-forming monomer or the matrix.

In some embodiments, the attachment moiety is capable of attaching covalently to the matrix-forming monomers and/or to the matrix. In some embodiments, the attachment moiety is capable of attaching covalently to a substrate for placing the biological sample on (e.g., glass slide) in addition to the matrix-forming monomers and/or to the matrix.

In some embodiments, the attachment moiety comprises an electrophilic group that is capable of interacting with a reactive nucleophilic group present on the matrix-forming monomers or the matrix to provide a covalent bond between the attachment moiety and the matrix-forming monomers or the matrix. In some embodiments, the nucleophilic groups on the matrix-forming monomers or the matrix having that capability include but are not limited to, sulfhydryl, hydroxyl and amino functional groups. In some embodiments, the attachment moiety comprises a maleimide, haloacetamide, or NHS ester.

In some embodiments, the attachment moiety comprises a nucleophilic group that is capable of interacting with a reactive electrophilic group present on the matrix-forming monomers or the matrix to provide a covalent bond between the attachment moiety and the matrix-forming monomers or the matrix. In some embodiments, the attachment moiety comprises or is a thiol, phenol, amino, hydrazide, hydroxylamine, hydrazine, thiosemicarbazone, hydrazine carboxylate, or arylhydrazide.

In some embodiments, the attachment moiety comprises a click functional group. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the attachment moiety can comprise any functional group involved in click reactions. In some embodiments, such click reactions may involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the attachment moiety comprises a click functional group, the matrix-forming monomer or the matrix to which it is capable of forming a covalent bond comprises the complementary click functional group to that of the attachment moiety. For example, in some embodiments, the attachment moiety comprises an azide moiety and the matrix-forming monomers or the matrix comprises a complementary alkyne moiety, or vice versa.

In some embodiments, the attachment moiety comprises a group capable of reacting with the matrix-forming monomers or the matrix. As detailed herein, matrix-forming monomers may include but are not limited to acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl(GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof. In some embodiments, the attachment moiety comprises an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, the attachment moiety comprises an alkenyl, allyl or vinyl moiety (e.g., —C═C— or HC—C— or HC═C—CH₂—), such as in N-(2-aminocthyl) methacrylamide, 2-aminoethyl methacrylate, 2-aminoethyl (E)-but-2-enoate, 2-aminocthyl methacrylate or methylacrylamide, or norbornene. Such alkenyl, allyl or vinyl moieties may be suitable for reaction with the matrix-forming monomer or the matrix.

In some embodiments, the attachment moiety comprises an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or malcimidyl moiety. In certain embodiments, the attachment moiety comprises a biotinyl moiety, dextrin moiety, a click moiety, a thiol moiety, norbornenyl moiety, furanyl moiety, alkyl ester moiety, or maleimidyl moiety.

In some embodiments, the formation of a bond between the attachment moiety and the matrix-forming monomers or the matrix is mediated by an external reagent or stimulus. For example, in some embodiments, the formation of a bond between the attachment moiety and the matrix-forming monomers or the matrix is initiated or induced by an enzyme, a catalyst, chemical reagents (e.g., acid, base, reducing agent, oxidant, etc.), heat, and/or light. In some embodiments, a covalent bond is formed between the attachment moiety and the matrix-forming monomers or the matrix. In some embodiments, contacting the matrix-forming monomers or the matrix with the attachment moiety further comprises contacting the matrix-forming monomers or the matrix with one or more reagents, and/or under suitable conditions to facilitate the formation of a covalent bond between the attachment moiety and the matrix-forming monomer or the matrix. For example, in some embodiments, such as wherein the attachment moiety comprises an alkene or a click functional group, the method may further comprise adding reagents to activate the alkene or click functional group, such as a radical initiator or a copper catalyst, respectively. In other embodiments, such as wherein the attachment moiety comprises an alkenyl, allyl or vinyl moiety, the method may further comprise exposing the matrix-forming monomer or the matrix and attachment moiety to ultraviolet (UV) light or heat to facilitate formation of a covalent bond. In some embodiments, such as wherein the attachment moiety comprises a norbornene moiety, furan moiety, maleimide moiety, or other alkenyl, allyl or vinyl moiety, the method may further comprise exposing the sample to light or heat. In yet other embodiments, the method may further comprise adding an enzyme to facilitate formation of a covalent bond. For example, in some embodiments, such as wherein the attachment moiety comprises a phenol moiety, the method may further comprise adding horseradish peroxidase (HRP).

In some embodiments, the attachment moiety is capable of attaching non-covalently to the matrix-forming monomers or the matrix. In some embodiments, the attachment moiety comprises a group capable of binding to the matrix-forming monomers or the matrix via non-covalent interaction, such as but not limited to hydrogen bonding, van der Waals interaction, and/or pi-stacking.

In some embodiments, the attachment to the matrix is non-covalent. In some embodiments, the attachment moiety is biotinylated. In some embodiments, the attachment moiety comprises a biotin moiety or a derivative thereof. In some embodiments, the attachment moiety is or comprises biotin and the anchoring moiety is or comprises streptavidin. In some embodiments, the attachment moiety is or comprises streptavidin and the anchoring moiety is or comprises biotin.

In some embodiments, the attachment moiety comprises a group capable of binding to an anchoring moiety in the matrix-forming monomers or the matrix. In some embodiments, the anchoring moiety comprises an acrylamide group, and the attachment moiety comprises a nucleophilic group capable of reacting with the acrylamide group of the anchoring moiety to form a covalent attachment. In some embodiments, the nucleophilic group capable of reacting with the acrylamide group is an amine. In some embodiments, the attachment between the metal nanoparticles and the matrix-forming monomer or the matrix is achieved by click reaction or Michael addition.

In some embodiments, the attachment moiety is of Formula (I),

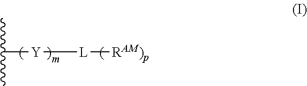

or a salt thereof,
wherein each Y is independently a group binding to the metal nanoparticles;
L is a bond or a linker moiety;
each R^{AM} is independently a group capable of forming a covalent or non-covalent bond with the matrix-forming monomer or the matrix;
m is an integer from 1 to 4;
p is an integer from 1 to 4; and
wherein the wavy line denotes the link of the attachment moiety to the metal nanoparticle.

In some embodiments, the attachment moiety (e.g., Formula (I)) comprises any of 1, 2, 3, or 4 groups binding with the metal nanoparticle (e.g., Y in Formula (I)). The Y of Formula (I) are each independently selected and can be any of the group binding to the metal nanoparticles described herein. In some embodiments, the attachment moiety is a bifunctional moiety wherein m is 1 and p is 1.

In some embodiments of Formula (I), p is any of 1, 2, 3, or 4. In some embodiments, the attachment moiety (e.g., Formula (I)) comprises more than one groups R^{AM} capable of forming a covalent or non-covalent bond with the matrix-forming monomer or the matrix (e.g., p is any of 2, 3, or 4), wherein each R^{AM} is independently selected from the embodiments provided herein, provided the more than one Y and R^{AM} groups are chemically compatible and have chemically compatible ribonucleic-binding mechanisms or reactions. In some embodiments, the R^{AM} of Formula (I) are each independently selected and can be any of the R^{AM} described herein.

In some embodiments, R^{AM} is capable of reacting with the matrix-forming monomer or the matrix to form a covalent bond. In some embodiments, R^{AM} is capable of reacting with the matrix-forming monomer or the matrix to form a covalent bond. In some embodiments, R^{AM} is a maleimide, haloacetamide, or NHS ester. In some embodiments, R^{AM} is a thiol, phenol, amino, hydrazide, hydroxylamine, hydrazine, thiosemicarbazone, hydrazine carboxylate, or arylhydrazide. In some embodiments, R^{AM} is a click functional group. In some embodiments, R^{AM} is an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, R^{AM} is a biotin moiety or a derivative thereof. In some embodiments, R^{AM} is an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In certain embodiments, R^{AM} is a biotinyl moiety, dextrin moiety, a click moiety, a thiol moiety, norbornenyl moiety, furanyl moiety, alkyl ester moiety, or maleimidyl moiety.

In some embodiments, the compound of formula (I) is a compound of formula (I-a). In some embodiments, the attachment moiety is a of Formula (I-a),

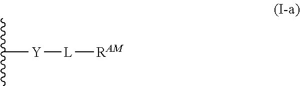

or a salt thereof,
wherein Y is a group binding to the metal nanoparticles;
L is a bond or a linker moiety;
R^{AM} is a group capable of forming a covalent or non-covalent bond with the matrix-forming monomer or the matrix; and
wherein the wavy line denotes the link of the attachment moiety to the metal nanoparticle.

In some embodiments, the compound of formula (I) is a compound of formula (I-b). In some embodiments, the attachment moiety is of Formula (I-b),

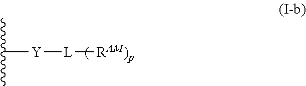

or a salt thereof,
wherein Y is a group binding to the metal nanoparticles;
L is a bond or a linker moiety;
each R^{AM} is independently a group capable of forming a covalent or non-covalent bond with the matrix-forming monomer or the matrix;
p is 1, 2, 3, or 4; and
wherein the wavy line denotes the link of the attachment moiety to the metal nanoparticle.

In some embodiments, the compound of formula (I) is a compound of formula (I-c). In some embodiments, the attachment moiety is of Formula (I-c),

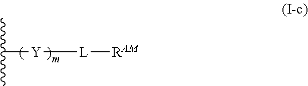

or a salt thereof,
wherein each Y is independently a group binding to the metal nanoparticles;
L is a bond or a linker moiety;
R^{AM} is a group capable of forming a covalent or non-covalent bond with the matrix-forming monomer or the matrix;
m is 1, 2, 3, or 4; and
wherein the wavy line denotes the link of the attachment moiety to the metal nanoparticle.

In some embodiments of Formula (I), L is a bond. In some embodiments of Formula (I), L is a linking moiety. In some embodiment, L is an unbranched or branched C₁-C₁₅₀ alkylene, which can be interrupted by 1 to 50 independently selected O, NH, N, S, C₆-C₁₂ arylene, or 5- to 12-membered heteroarylene. In some embodiments, L is an unbranched and uninterrupted C₁-C₁₅₀ alkylene. In some embodiments, L is a branched and uninterrupted C₁-C₁₅₀ alkylene. In some embodiments, L is an unbranched C₁-C₁₅₀ alkylene interrupted by 1 to 50 NH, O, or S. In some embodiments, L is

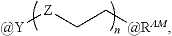

wherein @Y denotes the link of L to Y, and @R^{AM} denotes the link of L to R^{AM}, Z is CH₂, O, S; or NH; and n is an integer between 0 and 50. In some embodiments, L is

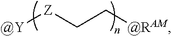

wherein @Y denotes the link of L to Y, and @R^{AM} denotes the link of L to R^{AM}, Z is CH₂, O, S; or NH; and n is an integer between 1 and 10. In some embodiment, L is

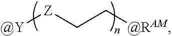

wherein @Y denotes the link of L to Y, and @R^{AM} denotes the link of L to R^{AM}, Z is CH₂, O, S; or NH; and n is 6. In some embodiments, L is an unbranched C₁-C₁₅₀ alkylene interrupted by 1 to 50 oxygen. In some embodiments, L comprises a polyethylene glycol portion or is a polyethylene glycol moiety. In some embodiments, L is

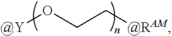

wherein @Y denotes the link of L to Y, and @R^{AM} denotes the link of L to R^{AM}, and n is an integer between 0 and 50. In some embodiments, L is

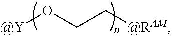

wherein @Y denotes the link of L to Y, and @R^{AM} denotes the link of L to R^{AM}, and n is an integer between 1 and 10. In some embodiments, L is

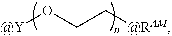

wherein @Y denotes the link of L to Y, and @R^{AM} denotes the link of L to R^{AM}, and n is 6. In some embodiments, L comprises an oligoethylene glycol. In some embodiments, L is an oligoethylene glycol moiety. In some embodiments, L is a branched C₁-C₁₅₀ alkylene interrupted by 1 to 50 oxygen. In some embodiments, L is an unbranched C₁-C₁₅₀ alkylene interrupted by 1 to 50 sulfurs. In some embodiments, L is

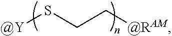

wherein @Y denotes the link of L to Y, and @R^{AM} denotes the link of L to R^{AM}, and n is an integer between 0 and 50. In some embodiment, L is

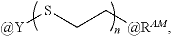

wherein @Y denotes the link of L to Y, and @R^{AM} denotes the link of L to R^{AM}, and n is 6. In some embodiments, L is a branched C₁-C₁₅₀ alkylene interrupted by 1 to 50 sulfurs. In some embodiments, L is a branched C₁-C₁₅₀ alkylene interrupted by 1 to 50 —NH—. In some embodiments, L is an unbranched C₁-C₁₅₀ alkylene interrupted by 1 to 50 —NH—. In some embodiments, L is

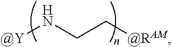

wherein @Y denotes the link of L to Y, and @R^{AM} denotes the link of L to R^{AM}, and n is an integer between 0 and 50. In some embodiment, L is

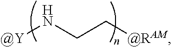

wherein @Y denotes the link of L to Y, and @R^{AM} denotes the link of L to R^{AM}, and n is 6. In some embodiments, L is a branched C₁-C₁₅₀ alkylene interrupted by 1 to 50 —NH—, wherein the —NH— is not at a branching point. In some embodiments, L is a branched C₁-C₁₅₀ alkylene interrupted by 1 to 50-N—, wherein the —N— is at a branching point. In some embodiments, L is an unbranched or branched C₁-C₁₅₀ alkylene interrupted by 1 to 50 independently selected C₆-C₁₂ arylene, for example, any of phenyl or naphthalene. In some embodiments, L is an unbranched or branched C₁-C₁₅₀ alkylene interrupted by 1 to 50 independently selected 5- to 12-membered heteroarylene, for example, any of pyridine, furan, pyrrole, or thiophene.

In some embodiments, Y is —S—. In some embodiments, Y is —O—.

In some embodiments, the attachment moiety is of Formula (I),

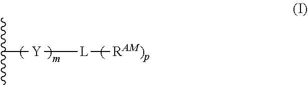

or a salt thereof,
wherein each Y is independently a group binding to the metal nanoparticles;
L is an unbranched or branched C₁-C₁₅₀ alkylene optionally interrupted by 1 to 50 heteroatoms independently selected from the group consisting of O, S and NH;
each R^{AM} is independently a group capable of forming a covalent or non-covalent bond with
the matrix-forming monomer or the matrix;
m is an integer from 1 to 4;
p is an integer from 1 to 4; and
wherein the wavy line denotes the link of the attachment moiety to the metal nanoparticle.

In some embodiments, when Y is —O—, then L is a bond or an unbranched and uninterrupted C₁-C₁₅₀ alkylene.

In some embodiments, the attachment moiety is a compound of Formula (I),

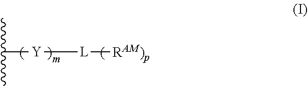

or a salt thereof,
wherein each Y is independently —S— or —O—;
L is a bond, an unbranched and uninterrupted C₁-C₁₅₀ alkylene,

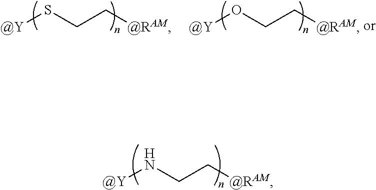

wherein @Y denotes the link of L to Y, and @R^{AM} denotes the link of L to R^{AM}, and wherein n is an integer from 1 to 50;
each R^{AM} is independently an acrylate moiety, methacrylate moiety, acrylamide moiety, methacrylamide moiety, biotinyl moiety, dextrin moiety, a click moiety, a thiol moiety, an amino moiety, norbornenyl moiety, furanyl moiety, alkyl ester moiety, or maleimidyl moiety;
m is an integer from 1 to 4;
p is an integer from 1 to 4; and
wherein the wavy line denotes the link of the attachment moiety to the metal nanoparticle.

In some embodiments, the attachment moiety is a compound of Formula (II-a),

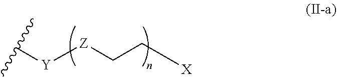

or a salt thereof, wherein
wherein Z is CH₂ or O, Y is S or O, X is —NH₂ or —OH, and n is an integer between 1 to 50, and wherein the wavy line denotes the link of the attachment moiety to the nanoparticle.

In some embodiments, the attachment moiety is a compound of Formula (xxxxx):

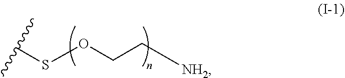

wherein n is an integer between 1-50, and wherein the wavy line denotes the link of the attachment moiety to the nanoparticle.

In some embodiments, the attachment moiety is capable of attaching covalently to an anchoring moiety in the matrix-forming monomer or the matrix. In some embodiments, the attachment moiety is capable of attaching covalently to an anchoring moiety in the matrix-forming monomers. In some embodiments, the attachment moiety is capable of attaching covalently to an anchoring moiety in the matrix. In some embodiments, the attachment moiety is capable of attaching non-covalently to an anchoring moiety in the matrix-forming monomers or the matrix. In some embodiments, the attachment moiety is capable of attaching non-covalently to an anchoring moiety in the matrix-forming monomers. In some embodiments, the attachment moiety is capable of attaching non-covalently to an anchoring moiety in the matrix.

It should be recognized that the attachment moiety and the anchoring moiety in the matrix-forming monomers or the matrix are selected with respect to one another, such that the anchoring moiety in the matrix-forming monomers or the matrix comprises a functional group capable of being attached to (e.g. ligating or bonding to) the attachment moiety. In some embodiments, the anchoring moiety in the matrix-forming monomer or the matrix comprises a complementary functional group capable of being attached covalently or non-covalently to the attachment moiety. For example, in some embodiments wherein the attachment moiety comprises an amino group, the anchoring moiety in the matrix-forming monomer or the matrix comprises an acrylamide. In another example, in some embodiments wherein the attachment moiety comprises a biotin, the anchoring moiety in the matrix-forming monomer or the matrix comprises streptavidin.

In some embodiments, the matrix-forming monomers are capable of forming a three-dimensional polymerized matrix under suitable reaction conditions. Suitable matrix-forming monomers are described herein. For example, in some embodiments, the matrix-forming monomers comprise polyacrylamide, cellulose, alginate, polyamide, cross-linked agarose, cross-linked dextran or cross-linked polyethylene glycol. In some embodiments, the method further comprises contacting the biological sample with matrix-forming monomers; and forming a three-dimensional polymerized matrix from the matrix-forming monomers, thereby embedding the biological sample in the three-dimensional polymerized matrix. In some embodiments, the metal nanoparticles are attached to the three-dimensional polymerized matrix. As detailed herein, in some embodiments, the step of contacting the biological sample and the matrix-forming monomers or the matrix further comprises contacting the biological sample with one or more reagents or under suitable conditions to facilitate the polymerization of the matrix-forming monomers to form the matrix.

In some embodiments, the three-dimensional polymerized matrix is formed by subjecting the matrix-forming monomer to polymerization. In some embodiments, the polymerization is initiated by adding a polymerization-inducing catalyst, UV light or functional cross-linkers. In some embodiments, the method further comprises staining, permeabilizing, cross-linking, expanding, and/or de-cross-linking the biological sample embedded in the three-dimensional polymerized matrix. In some embodiments, the method further comprises staining, permeabilizing, cross-linking, expanding, and/or de-cross-linking the biological sample embedded in the three-dimensional polymerized matrix after the attachment moiety has been covalently or non-covalently bonded to the matrix-forming monomer.

In some embodiments, the biological sample is cleared. In some embodiments, the biological sample can be cleared by any suitable method, such as with a detergent, a lipase, and/or a protease.

### III. Signal Generation, Detection, and Analysis

In some embodiments, provided herein is a method for sample analysis comprising providing a biological sample embedded in a hydrogel comprising a matrix that is attached to metal nanoparticles. In some embodiments, the method comprises generating a fluorescent signal associated with an analyte in the biological sample. In some embodiments, the method comprises detecting the fluorescent signal at a 3D location in the matrix which corresponds to the 3D location of the analyte in the biological sample.

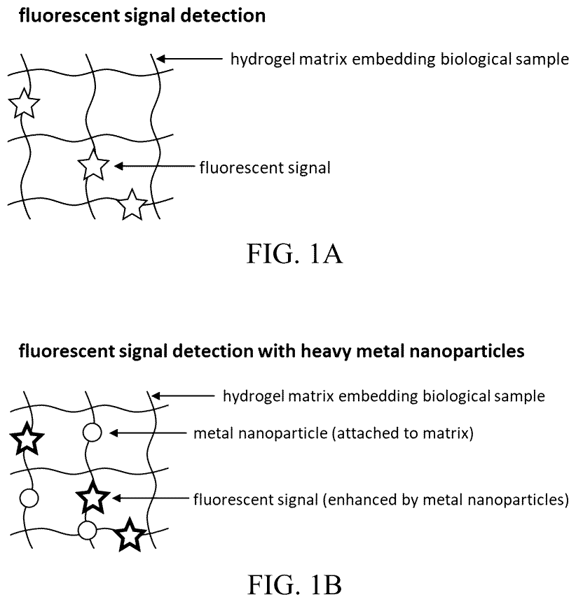

FIGS. 1A-1B show exemplary schematics illustrating detection of fluorescent signals in a matrix embedding a biological sample, without (FIG. 1A) or with (FIG. 1B) metal nanoparticles attached to the matrix. Fluorescent signals, shown as stars, are enhanced in the presence of metal nanoparticles, for example by metal-enhanced fluorescence (MEF).

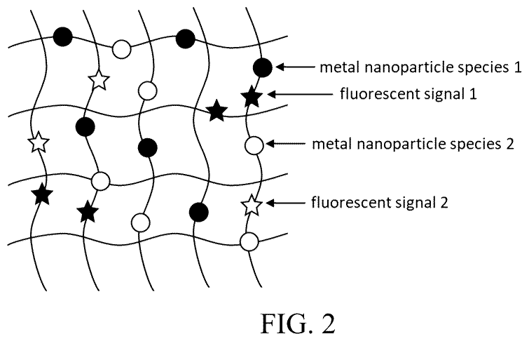

FIG. 2 shows an exemplary schematic illustrating fluorescent signals and metal nanoparticles of more than one species attached to a hydrogel matrix embedding a biological sample. The figure illustrates that different species of metal nanoparticles (e.g. metal nanoparticle species 1 and metal nanoparticle species 2) can be attached to the matrix. Different metal nanoparticle species may optimally enhance fluorescent signals in different wavelengths (e.g. a first and second fluorescent signal). Fluorescent signals in different wavelengths may be generated and detected in different 3D locations or the same 3D locations in the biological sample.

In some embodiments, the metal nanoparticles enhance the fluorescent signal. In some embodiments, the metal nanoparticles enhance the fluorescent signal via metal-enhanced fluorescence (MEF). In some embodiments, the enhancement of the fluorescent signal is an increase in the intensity of the fluorescent signal. In some embodiments, the enhancement of the fluorescent signal facilitates an increased ability to detect the analyte (e.g. an increased sensitivity of the method). For example, in some embodiments, the enhancement of the fluorescent signal allows for the detection of a larger number of analytes in the sample.

In some embodiments, the fluorescent signal of the analyte is enhanced by at least about 5%, such as at least about any of 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, or 500%, in comparison to a fluorescent signal generated by the same method in the absence of the metal nanoparticles. In some embodiments, the contrast between the fluorescent signal from the analyte of interest and the background autofluorescence is enhanced by at least about 5%, such as at least about any of 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, or 500%, in comparison to contrast observed using the same method in the absence of the metal nanoparticles. In some embodiments of the foregoing, the fluorescent signal associated with an analyte, and/or the contrast between the fluorescent signal associated with an analyte and the background autofluorescence is enhanced in one or more emission spectra. In some embodiments, the wavelength range of the one or more emission spectra is about 200 nm to about 1200 nm, such as about any of 200 nm to 1000 nm, 300 nm to 1000 nm, 400 nm to 1000 nm, or 400 nm to 800 nm.

In some embodiments, the relative location of the fluorescent signal and one or more metal nanoparticles covalently attached to the hydrogel is specified or configured such that a strong MEF effect can be observed. In some embodiments, the relative location of the fluorescent signal and one or more metal nanoparticles covalently attached to the hydrogel is specified or configured by providing a suitable concentration of metal nanoparticles in the 3D hydrogel such that a strong MEF effect can be observed. In some embodiments, the location of the fluorescent signal is between about 5 nm and about 90 nm from one or more metal nanoparticles covalently attached to the hydrogel.

In some aspects, generating the fluorescent signal associated with the analyte can be performed by any suitable method. In some embodiments, the analyte is a fluorescent analyte and the fluorescent signal is fluorescence of the analyte. For example, certain biological samples can include products of fluorescent transgenes (e.g. green fluorescent protein, or GFP). Thus, in some aspects, the fluorescent signal can be directly generated from the analyte, which is fluorescent. In other embodiments, the analyte is not itself substantially fluorescent, and any suitable method can be used to generate a fluorescent signal associated with the analyte (e.g. using probes).

In some embodiments, the analyte is a nucleic acid analyte. In some embodiments, the analyte is a non-nucleic acid analyte. In some embodiments, generating the fluorescent signal associated with the analyte comprises contacting the biological sample with a detectably labeled probe that binds directly or indirectly to the analyte or a product thereof, and using the detectably labeled probe to generate a fluorescent signal. In some embodiments, the detectably labeled probe comprises a fluorescent label. In some embodiments, the detectably labeled probe binds to a primary probe that directly binds to the analyte. In some embodiments, the detectably labeled probe binds to an intermediate probe that binds directly or indirectly to a primary probe that directly binds to the analyte. In some embodiments, any of the probes provided herein, such as the primary probe and/or intermediate probe are nucleic acid probes. In some embodiments, the binding of nucleic acid probes is mediated by hybridization. In some embodiments, the nucleic acid probes can have any suitable structure. For example, in some embodiments, the primary probe and the intermediate probe are each independently selected from the group consisting of: a probe comprising a 3′ or 5′ overhang, optionally wherein the 3′ or 5′ overhang comprises one or more barcode sequences; a probe comprising a 3′ overhang and a 5′ overhang, optionally wherein the 3′ overhang and the 5′ overhang each independently comprises one or more barcode sequences; a circular probe; a circularizable probe or probe set; a probe or probe set comprising a split hybridization region configured to hybridize to a splint, optionally wherein the split hybridization region comprises one or more barcode sequences; and a combination thereof.

In some embodiments, generating the fluorescent signal associated with the analyte comprises performing rolling circle amplification (RCA) to generate an RCA product (RCP). In some embodiments, the RCA is performed using as template a circular or circularized probe that is hybridized to the analyte, a product thereof, or to a probe bound directly or indirectly thereto. In some embodiments, any one or more of the analyte, the detectably labeled probe, the intermediate probe, the primary probe, and the RCP, is immobilized in the matrix.

In some embodiments, the attachment of the metal nanoparticles to the matrix allows for various analysis and/or processing steps to be performed while retaining the metal nanoparticles in the matrix. Thus, the metal nanoparticles can facilitate, for example, the enhancement of sequentially generated fluorescent signals. In some embodiments, the fluorescent signal is a first fluorescent signal, the detectably labeled probe is a first detectably labeled probe, and the method further comprises: contacting the biological sample with a subsequent detectably labeled probe that binds directly or indirectly to a subsequent analyte or product thereof in the biological sample and using the subsequent detectably labeled probe to generate a subsequent fluorescent signal; and detecting the subsequent fluorescent signal at a 3D location in the matrix which corresponds to the 3D location of the subsequent analyte in the biological sample. In some embodiments, the method comprises removing the first detectably labeled probe from the biological sample, optionally via one or more wash steps, prior to contacting the sample with the subsequent detectably labeled probe. In some embodiments, the metal nanoparticles remain attached to the matrix at least until detecting the subsequent fluorescent signal. Thus, it can be seen that the methods provided herein have the advantage of allowing the same metal nanoparticles to be used in multiple signal detection and/or processing steps that involve removal of other materials from the 3D hydrogel, thus reducing the cost of the method in comparison to methods involving providing metal nanoparticles in each of the multiple steps. In some embodiments, the metal nanoparticles enhance the subsequent fluorescent signal via metal-enhanced fluorescence. In some embodiments, the first analyte and the subsequent analyte are the same or different.

In some aspects, the biological sample can be processed to facilitate signal detection and/or analysis. In some embodiments, the biological sample is a cleared biological sample. In some embodiments, the biological sample is cleared at any suitable step, such as before and/or after matrix formation. In some embodiments, the biological sample is cleared before detecting the fluorescent signal. In some embodiments, the biological sample is cleared after the metal nanoparticles are attached to the matrix. In some embodiments, the biological sample is cleared after immobilizing the analyte or a product thereof, the detectably labeled probe, the first detectably labeled probe, the second detectably labeled probe, the primary probe, the intermediate probe, and/or the RCP in the matrix. Different advantages can be associated with clearing at different steps. For example, clearing the biological sample before matrix formation may reduce false positive signals when detecting the target nucleic acid. Clearing the biological sample after matrix formation may lower background signal.

In some aspects, the methods provided herein allow for analysis of samples that are 3-dimensional (3D), and for detection of fluorescent signals throughout the 3D volume of the sample, wherein the fluorescent signals are enhanced by metal nanoparticles. In some embodiments, the biological sample is provided on a solid support. In some embodiments, the biological sample is a tissue section. In some embodiments, the biological sample is or comprises an intact tissue section. In some embodiments, the biological sample is non-homogenized. In some embodiments, the biological sample is or comprises cells. In some embodiments, the biological sample is a cell pellet, cell block, or a section thereof. In some embodiments, the biological sample is non-homogenized and optionally selected from the group consisting of a formalin-fixed, paraffin-embedded (FFPE) sample, a frozen tissue sample, and a fresh tissue sample. In some embodiments, the biological sample is a section having a thickness of at least 0.2 μm, at least 1 μm, or at least 5 μm. In some aspects, the thickness of the biological sample is such that a metal surface adjacent to the biological sample (e.g. the surface of a slide on which the sample is mounted) would not be capable of enhancing fluorescent signals throughout the biological sample via metal-enhanced fluorescence (e.g. at a distance of greater than 90 nm from the surface). Thus, it can be seen that the methods provided herein have the advantage of allowing for enhancement of fluorescent signals throughout a 3D volume by MEF that would not be possible by other methods, such as those comprising providing metal nanoparticles on a 2D surface.

In some aspects, provided herein is a method comprising detection of one or more RCA products (RCPs) by generating fluorescent signals in sequential steps, wherein the fluorescent signals generated in sequential steps are each enhanced by the same metal nanoparticles. For example, in some embodiments, provided herein is a method for sample analysis, comprising: providing a biological sample embedded in a hydrogel comprising a matrix that is attached to metal nanoparticles; contacting the sample with a circular probe that hybridizes to an analyte or contacting the sample with a circularizable probe that hybridizes to the analyte and circularizing the circularizable probe to form a circularized probe; performing rolling circle amplification (RCA) using the circular or circularized probe as template to generate an RCA product (RCP); contacting the biological sample with a first detectably labeled probe that binds directly or indirectly to the RCP and detecting a fluorescent signal generated from the first detectably labeled probe at a 3-dimensional (3D) location in the matrix which corresponds to the 3D location of the analyte in the biological sample; removing the first detectably labeled probe from the biological sample, optionally via one or more wash steps; and contacting the biological sample with a subsequent detectably labeled probe that binds directly or indirectly to the RCP and detecting a subsequent fluorescent signal generated from the subsequent detectably labeled probe at a 3-dimensional (3D) location in the matrix which corresponds to the 3D location of the analyte in the biological sample. In some embodiments, the metal nanoparticles remain attached to the matrix at least until detecting the subsequent fluorescent signal. In some embodiments, the metal nanoparticles enhance the first fluorescent signal and subsequent fluorescent signal via metal-enhanced fluorescence (MEF).

In some aspects, the provided methods involve analyzing, e.g., detecting or determining, one or more sequences present in the analytes or provided probes or products thereof (e.g., rolling circle amplification products thereof). In some embodiments, generating/detecting the fluorescent signal is performed at one or more locations (e.g. 3D locations) in the biological sample and/or matrix (e.g. matrix embedding the biological sample). In some embodiments, the locations are, and/or correspond to, the locations of target analytes, such as nucleic acids, in the biological sample.

In some embodiments, generating/detecting the fluorescent signals comprises a plurality of repeated cycles of hybridization and removal of probes (e.g., detectably labeled probes, or intermediate probes that bind to detectably labeled probes). In some embodiments, the hybridization is to a primary probe hybridized to the target nucleic acid, or to a product (such as a rolling circle amplification product) generated from the probe hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and can be detected in one or more hybridization cycles (e.g., sequential hybridization assays, or sequencing by hybridization).

In some embodiments, the generating and/or detecting the fluorescent signal can comprise binding an intermediate probe directly or indirectly to the primary probe, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized primary probe as a template. In some embodiments, the method comprises detecting a rolling circle amplification product (RCP) generated using a circular or circularized probe or probe set that binds to a primary probe as a template. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the primary probes or the products of the primary probes.

In some embodiments, generating/detecting the fluorescent signal can comprise: detecting signals associated with detectably labeled probes that are hybridized to barcode regions or complements thereof in a primary probe or a product thereof (e.g., an RCP); and/or detecting signals associated with detectably labeled probes that are hybridized to intermediate probes which are in turn hybridized to the barcode regions or complements thereof. In some embodiments, the detectably labeled probes can be fluorescently labeled.

In some embodiments, the methods comprise detecting the sequence in all or a portion of a primary probe or an RCP, or detecting a sequence of a primary probe or RCP, such as one or more barcode sequences present in the primary probe or RCP. In some embodiments, the sequence of the RCP is indicative of a sequence of the target nucleic acid to which the RCP is hybridized. In some embodiments, the analysis and/or sequence determination comprises in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, generating and/or detecting the fluorescent signal comprises hybridizing to the probe directly or indirectly an oligonucleotide labeled with a fluorophore, an isotope, a mass tag, or a combination thereof. In some embodiments, the method comprises imaging the probe hybridized to the target nucleic acid (e.g., imaging one or more detectably labeled probes hybridized thereto).

In some instances, the disclosed methods may comprise assembly of branched hybridization complexes. For example, the methods may comprise the use of a branched DNA (bDNA) amplification approach to amplify signals. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (Xia, et al. (2019), “Multiplexed Detection of RNA Using MERFISH and Branched DNA Amplification”, Scientific Reports 9:7721, which is hereby incorporated herein in its entirety). The degree of amplification in bDNA amplification is controlled by the design of the amplification reaction, e.g., the assembled bDNA structures cannot grow indefinitely even in the presence of excess reagents, which may be used to control spot size or limit the variability in brightness from molecule to molecule (Xia, et al. (2019), ibid.).

In some instances, the disclosed methods may comprise the use of a hybridization chain reaction (HCR) approach to amplify signals. HCR is an enzyme-free nucleic acid amplification based on a triggered chain of hybridization of nucleic acid molecules starting from HCR monomers, which hybridize to one another to form a nicked double-stranded nucleic acid polymer. This polymer is the product of the HCR reaction which is ultimately detected in order to indicate the presence of the target analyte. HCR is described in detail in Dirks and Pierce, 2004, PNAS, 101 (43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28 (11), 1208-1212; and Song et al, 2012, Analyst, 137, 1396-1401), the content of each which is herein incorporated by reference in its entirety. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction can be carried out with more than two species or types of HCR monomers. For example, a system involving three HCR monomers can be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers can readily be conceived.

In some embodiments of HCR, two fluorescently-labeled metastable hairpin oligonucleotides self-assemble into long fluorescent polymers starting from an initiator sequence present on each probe molecule (Xia, et al. (2019), ibid.). The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, similar to HCR reactions that use hairpin monomers, linear oligo hybridization chain reaction (LO-HCR) can also be used for signal amplification. In some embodiments, an initiator is contacted with a plurality of LO-HCR monomers of at least a first and a second species to generate a polymeric LO-HCR product that can be detected. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure.

In some embodiments, the methods provided herein can comprise signal amplification by performing a primer exchange reaction (PER), for instance, on a primary probe or an RCP. In various embodiments, a primer with a domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising primary barcode sequences in hybridized and ligated primary immobilizable probes as described herein. See e.g., U.S. Pat. Pub. No. US20190106733, which is incorporated herein by reference in its entirety, for exemplary molecules and PER reaction components.

In some aspects, the provided methods comprise imaging a detectably labeled probe bound directly or indirectly to a primary probe or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urcase.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycocrythrin.

Examples of bioluminescent markers comprise, but are not limited to, luciferase (e.g., bacterial, firefly and click beetle), luciferin, acquorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345, each of which is incorporated by reference herein in its entirety).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, which is incorporated by reference herein in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for generation/detection of the fluorescent signal. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for generation/detection of the fluorescent signal. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343 (6177), 1360-1363.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, the method comprises contacting the biological sample with probes comprising barcode sequences, which may correspond to target analytes. Thus, detection of the barcode sequences can reveal the presence of the target analytes. In some embodiments, the method comprises sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences or complements thereof by detecting the corresponding sequences of signal codes detected from sequential hybridization, detection, and removal of sequential pools of intermediate probes and detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization, for example as described in US 2021/0340618, the content of which is herein incorporated by reference in its entirety.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization can be used for sequencing. Such methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, each of which is incorporated by reference herein in its entirety.

In some embodiments, the analysis and/or sequence determination involves washing to remove unbound polynucleotides, thereafter revealing a fluorescent product for imaging.

### IV. Samples, Analytes, and Target Sequences

### A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular probe or probe set or circularizable probe or probe set (e.g., padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., padlock probe).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DIR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, cosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and cosin (H&E).

The sample can be stained using hematoxylin and cosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel matrix) can be isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes in a single biological sample are provided.

### B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. Sec, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed. In some embodiments, the products described herein can be detected using fluorescent signals enhanced by the metal nanoparticles attached to the hydrogel matrix embedding biological sample (e.g., as described in Sections II and III).

### (a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a circularizable probe or probe set (e.g., padlock probe), a gapped circularizable probe or probe set (e.g., gapped padlock probe), a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and probe parts that are configured to be ligated to form a ligated probe using RNA as ligation template. The specific probe or probe set design can vary.

### (b) Ligation

In some embodiments, a product of an endogenous analyte and/or a labeling agent is a ligation product. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set capable of DNA-templated ligation, such as from a cDNA molecule. Sec, e.g., U.S. Pat. No. 8,551,710, which is hereby incorporated by reference in its entirety. In some embodiments, provided herein is a probe or probe set capable of RNA-templated ligation. Sec, e.g., U.S. Pat. Pub. 2020/0224244 which is hereby incorporated by reference in its entirety. In some embodiments, the probe set is a SNAIL probe set. Scc, e.g., U.S. Pat. Pub. 20190055594, which is hereby incorporated by reference in its entirety. In some embodiments, provided herein is a multiplexed proximity ligation assay. Sec, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. Sec, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76 (14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° NTM DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, a circularizable probe or probe set (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, a product is a primer extension product of an analyte, a labeling agent, a probe or probe set bound to the analyte (e.g., a circularizable probe or probe set such as padlock probe bound to genomic DNA, mRNA, or cDNA), or a probe or probe set bound to the labeling agent (e.g., a circularizable probe or probe set such as padlock probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some aspects, a primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 ((29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2019/0241950, US 2016/0024555, US 2018/0251833 and US 2017/0219465, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination.

### C. Target Sequences

A target sequence for a probe disclosed herein may be comprised in or associated with any analyte disclose herein, including an endogenous analyte (e.g., a viral or cellular nucleic acid), a labeling agent, or a product of an endogenous analyte and/or a labeling agent.

In some embodiments described herein, the analyte comprises or is associated with a target sequence. In some embodiments, a target sequence for a nucleic acid probe described herein is a marker sequence for a given analyte. A marker sequence is a sequence that identifies a given analyte (e.g., alone or in combination with one or more other marker sequences). Thus, in some embodiments, a marker sequence for a given target analyte is specific to that analyte, or unique, such that multiple target analytes can be distinguished from each other.

A “marker sequence” is thus a sequence which marks, is associated with, or identifies a given analyte. It is a sequence by which a given analyte may be detected and distinguished from other analytes. Where an “analyte” comprises a group of related molecules e.g. isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g. a particular variant. In this way different variants, or isoforms, or mutants may be identified or distinguished from one another.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte or labeling agent (e.g., any of products described in Section B (iii) above) as a tag or identifier (ID) sequence (e.g. a barcode) for the analyte or labeling agent. It may thus be a synthetic or artificial sequence.

In some embodiments, an endogenous analyte, labeling agent, or a product of an analyte or labeling agent may comprise multiple copies of the target sequence. For example, a probe molecule, or probe component, may comprise multiple copies of a target sequence. In another example, an amplification product may be generated which comprises multiple copies of the target sequence (e.g., multiple copies of a barcode sequence).

It will be understood that in the case of an analyte, product, or labeling agent comprising multiple target sequences, while each of the target sequences may comprise a binding site for a nucleic acid probe described herein, in practice not all of these binding sites may (or will) be occupied by a nucleic acid probe after nucleic acid probe hybridization. In some embodiments, it suffices that a number, or multiplicity, of such binding sites are bound by a nucleic acid probe. Thus, in some embodiments the nucleic acid probe may hybridize to at least one target sequence present in an analyte, labeling agent, or product of an analyte or labeling agent. In some embodiments, the nucleic acid probe hybridizes to multiple target sequences present in the analyte, labeling agent, or product of an analyte or labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### V. Compositions and Kits

In some aspects, provided herein are compositions comprising any of the biological samples, hydrogel matrices or components thereof, metal nanoparticles, and/or probes (e.g. detectably labeled probe, intermediate probe, primary probe) provided herein.

Also provided herein are kits for analyzing a sample according to any of the methods described herein. In some embodiments, the kit comprises any of the biological samples, hydrogel matrices, metal nanoparticles, and/or probes (e.g. detectably labeled probe, intermediate probe, primary probe) provided herein. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer, ligation buffer, and/or other reagents. In some embodiments, the kits contain reagents for detection and/or sequencing, such as barcode detection probes or detectable labels. In some embodiments, the kits optionally contain other components, for example nucleic acid primers (e.g. RCA primers).

In some aspects, provided herein is a kit for analyzing a biological sample, the kit comprising: a) a probe configured to bind directly or indirectly to a target analyte in the biological sample, wherein the probe comprises a fluorescent label; and b) hydrogel matrix forming monomers comprising metal nanoparticles, wherein the metal nanoparticles are capable of enhancing, via metal-enhanced fluorescence (MEF), a fluorescent signal generated from the fluorescent label.

In some aspects, provided herein is a kit for analyzing a biological sample, the kit comprising: a) a probe configured to bind directly or indirectly to a target analyte in the biological sample, wherein the probe comprises a fluorescent label; b) hydrogel matrix forming monomers; and c) metal nanoparticles configured to be attached to the hydrogel matrix forming monomers and/or to a hydrogel matrix formed from the hydrogel matrix forming monomers, wherein the metal nanoparticles are capable of enhancing, via metal-enhanced fluorescence (MEF), a fluorescent signal generated from the fluorescent label.

In some aspects, provided herein is a system for analyzing a biological sample, the system comprising: a) a probe configured to bind directly or indirectly to a target analyte in the biological sample, wherein the probe comprises a fluorescent label; and b) hydrogel matrix forming monomers comprising metal nanoparticles, wherein the metal nanoparticles are capable of enhancing, via metal-enhanced fluorescence (MEF), a fluorescent signal generated from the fluorescent label.

In some aspects, provided herein is a system for analyzing a biological sample, the system comprising: a) a probe configured to bind directly or indirectly to a target analyte in the biological sample, wherein the probe comprises a fluorescent label; b) hydrogel matrix forming monomers; and c) metal nanoparticles configured to be attached to the hydrogel matrix forming monomers and/or to a hydrogel matrix formed from the hydrogel matrix forming monomers, wherein the metal nanoparticles are capable of enhancing, via metal-enhanced fluorescence (MEF), a fluorescent signal generated from the fluorescent label.

### VI. Opto-Fluidic Instruments for Analysis of Biological Samples

In some aspects, provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section V). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules by detecting the generated signals (e.g., as described in Section III) in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

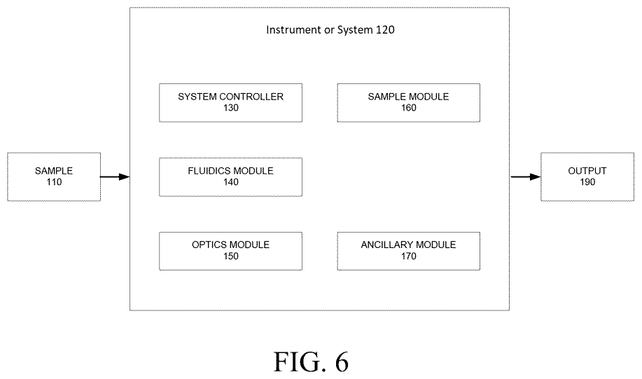

FIG. 6 shows an example workflow of analysis of a biological sample 110 (e.g., cell or tissue sample) using an instrument or system (e.g., an opto-fluidic instrument) 120, according to various embodiments. In various embodiments, the sample 110 can be a biological sample (e.g., a tissue) that includes molecules such as DNA, RNA, proteins, antibodies, etc. For example, the sample 110 can be a sectioned tissue that is treated to access the RNA thereof for labeling with probes described herein (e.g., in Section II). Ligation of the probes may generate a circular probe which can be enzymatically amplified and bound with detectably labeled probes, which can create bright signal that is convenient to image and has a high signal-to-noise ratio.

In various embodiments, the sample 110 may be placed in the instrument or system (e.g., opto-fluidic instrument) 120 for analysis and detection of the molecules in the sample 110. In various embodiments, the instrument or system (e.g., opto-fluidic instrument) 120 can be a system configured to facilitate the experimental conditions conducive for the detection of the target molecules. For example, the instrument or system (e.g., opto-fluidic instrument) 120 can include a fluidics module 140, an optics module 150, a sample module 160, and an ancillary module 170, and these modules may be operated by a system controller 130 to create the experimental conditions for the probing of the molecules in the sample 110 by selected probes (e.g., circularizable DNA probes), as well as to facilitate the imaging of the probed sample (e.g., by an imaging system of the optics module 150). In various embodiments, the various modules of the instrument or system (e.g., opto-fluidic instrument) 120 may be separate components in communication with each other, or at least some of them may be integrated together.

In various embodiments, the sample module 160 may be configured to receive the sample 110 into the instrument or system (e.g., opto-fluidic instrument) 120. For instance, the sample module 160 may include a sample interface module (SIM) that is configured to receive a sample device (e.g., cassette) onto which the sample 110 can be deposited. That is, the sample 110 may be placed in the instrument or system (e.g., opto-fluidic instrument) 120 by depositing the sample 110 (e.g., the sectioned tissue) on a sample device that is then inserted into the SIM of the sample module 160. In some instances, the sample module 160 may also include an X-Y stage onto which the SIM is mounted. The X-Y stage may be configured to move the SIM mounted thereon (e.g., and as such the sample device containing the sample 110 inserted therein) in perpendicular directions along the two-dimensional (2D) plane of the instrument or system (e.g., opto-fluidic instrument) 120.

The experimental conditions that are conducive for the detection of the molecules in the sample 110 may depend on the target molecule detection technique that is employed by the instrument or system (e.g., opto-fluidic instrument) 120. For example, in various embodiments, the instrument or system (e.g., opto-fluidic instrument) 120 can be a system that is configured to detect molecules in the sample 110 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 110 using reagents such as washing/stripping reagents, hybridizing reagents, etc., and such reagents may be provided by the fluidics module 140.

In various embodiments, the fluidics module 140 may include one or more components that may be used for storing the reagents, as well as for transporting said reagents to and from the sample device containing the sample 110. For example, the fluidics module 140 may include reservoirs configured to store the reagents, as well as a waste container configured for collecting the reagents (e.g., and other waste) after use by the instrument or system (e.g., opto-fluidic instrument) 120 to analyze and detect the molecules of the sample 110. Further, the fluidics module 140 may also include pumps, tubes, pipettes, etc., that are configured to facilitate the transport of the reagent to the sample device (e.g., and as such the sample 110). For instance, the fluidics module 140 may include pumps (“reagent pumps”) that are configured to pump washing/stripping reagents to the sample device for use in washing/stripping the sample 110 (e.g., as well as other washing functions such as washing an objective lens of the imaging system of the optics module 150).

In various embodiments, the ancillary module 170 can be a cooling system of the instrument or system (e.g., opto-fluidic instrument) 120, and the cooling system may include a network of coolant-carrying tubes that are configured to transport coolants to various modules of the instrument or system (e.g., opto-fluidic instrument) 120 for regulating the temperatures thereof. In such cases, the fluidics module 140 may include coolant reservoirs for storing the coolants and pumps (e.g., “coolant pumps”) for generating a pressure differential, thereby forcing the coolants to flow from the reservoirs to the various modules of the instrument or system (e.g., opto-fluidic instrument) 120 via the coolant-carrying tubes. In some instances, the fluidics module 140 may include returning coolant reservoirs that may be configured to receive and store returning coolants, e.g., heated coolants flowing back into the returning coolant reservoirs after absorbing heat discharged by the various modules of the instrument or system (e.g., opto-fluidic instrument) 120. In such cases, the fluidics module 140 may also include cooling fans that are configured to force air (e.g., cool and/or ambient air) into the returning coolant reservoirs to cool the heated coolants stored therein. In some instance, the fluidics module 140 may also include cooling fans that are configured to force air directly into a component of the instrument or system (e.g., opto-fluidic instrument) 120 so as to cool said component. For example, the fluidics module 140 may include cooling fans that are configured to direct cool or ambient air into the system controller 130 to cool the same.

As discussed above, the instrument or system (e.g., opto-fluidic instrument) 120 may include an optics module 150 which include the various optical components of the instrument or system (e.g., opto-fluidic instrument) 120, such as but not limited to a camera, an illumination module (e.g., LEDs), an objective lens, and/or the like. The optics module 150 may include a fluorescence imaging system that is configured to image the fluorescence emitted by the probes (e.g., oligonucleotides) in the sample 110 after the probes are excited by light from the illumination module of the optics module 150.

In some instances, the optics module 150 may also include an optical frame onto which the camera, the illumination module, and/or the X-Y stage of the sample module 160 may be mounted.

In various embodiments, the system controller 130 may be configured to control the operations of the instrument or system (e.g., opto-fluidic instrument) 120 (e.g., and the operations of one or more modules thereof). In some instances, the system controller 130 may take various forms, including a processor, a single computer (or computer system), or multiple computers in communication with each other. In various embodiments, the system controller 130 may be communicatively coupled with data storage, set of input devices, display system, or a combination thereof. In some cases, some or all of these components may be considered to be part of or otherwise integrated with the system controller 130, may be separate components in communication with each other, or may be integrated together. In other examples, the system controller 130 can be, or may be in communication with, a cloud computing platform.

In various embodiments, the instrument or system (e.g., opto-fluidic instrument) 120 may analyze the sample 110 and may generate the output 190 that includes indications of the presence of the target molecules in the sample 110. For instance, with respect to the example embodiment discussed above where the instrument or system (e.g., opto-fluidic instrument) 120 employs a hybridization technique for detecting molecules, the instrument or system (e.g., opto-fluidic instrument) 120 may cause the sample 110 to undergo successive rounds of detectably labeled probe hybridization (e.g., using two or more sets of fluorescent probes, where each set of fluorescent probes is excited by a different color channel) and be imaged to detect target molecules in the probed sample 110. In such cases, the output 190 may include optical signatures (e.g., a codeword) specific to each gene, which allow the identification of the target molecules.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Metal-Enhanced Fluorescence of Fluorescent Signals Corresponding to Analytes in a Biological Sample

This Example provides an exemplary method for analyzing a biological sample wherein metal-enhanced fluorescence (MEF) was used to enhance fluorescent signals corresponding to analytes in situ in a biological sample.

Formalin-fixed mouse brain sections were mounted on glass slides or gold-coated slides. The samples were contacted with circularizable probes (e.g. padlock probes) targeting nucleic acid analytes corresponding to transcripts of Satb2 (cortex-specific expression), Prox1 (dentate gyrus-specific expression) or Plp1 (cell type-specific expression), or negative control circularizable probes not targeting specific analytes in the sample. The circularizable probes were hybridized to the respective target nucleic acid analytes in the biological sample and ligated to generate circularized probes.

The circularized probes were used as templates for rolling circle amplification (RCA). An RCA primer was hybridized to the circularized probes, and an RCA reaction mixture (containing Phi29 reaction buffer, dNTPs, and Phi29 polymerase) was added to the sample. The sample was incubated at 37° C. for 3 hours to allow the RCA to proceed, thereby generating rolling circle amplification products (RCPs) corresponding to the target analytes, and then the RCA was terminated.

RCPs were hybridized to different detectable probes labeled with fluorophores corresponding to each of the analytes, as follows: AF750 for Satb2; Cy3 for Prox1; AF488 for Plp1; Cy5 for negative control circularizable probes. The samples were imaged by fluorescence microscopy to detect fluorescent signals corresponding to each of the transcripts.

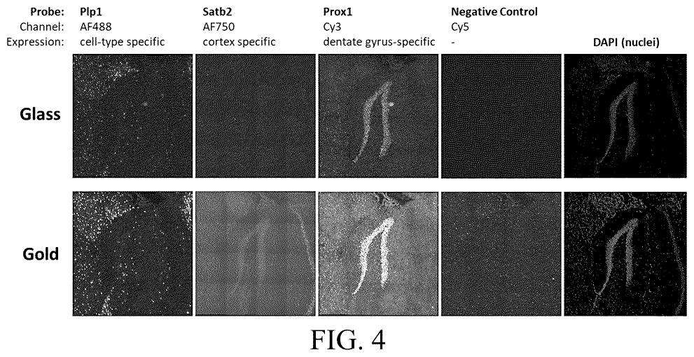

FIG. 4 shows representative resulting fluorescent images of the biological samples mounted on glass or gold-coated slides. The two rows of images are from glass or gold-coated slides, and each column shows a fluorescent image obtained in the indicated channel. The figure shows that samples mounted on gold-coated slides exhibited enhancement of fluorescent signals in comparison to samples mounted on glass slides.

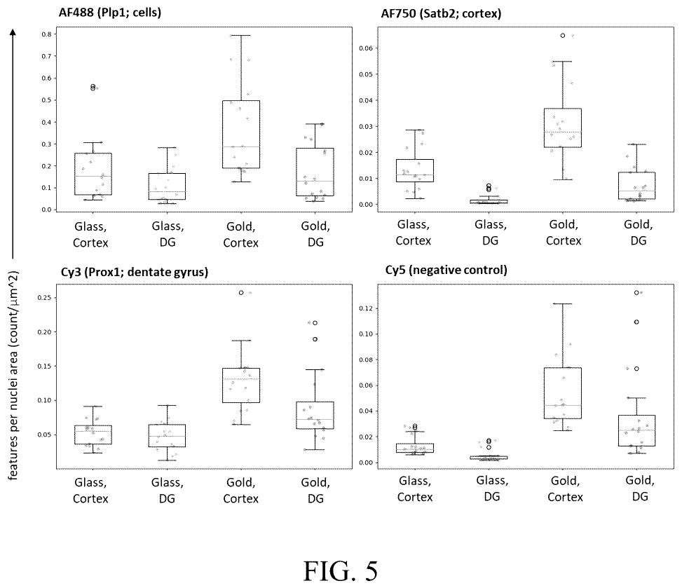

FIG. 5 shows quantification of the number of individual fluorescent signals (features) detected per nuclei area in cortex or dentate gyrus of samples on glass or gold-coated slides, in channels corresponding to indicated transcripts or negative control. The figure shows that gold coated slides enhanced assay sensitivity when detecting fluorescent signals in situ.

Taken together, the data show that metal-enhanced fluorescence (MEF) can be used to enhance fluorescent signals associated with analytes in situ in a biological sample, including for signals generated from target nucleic acids using RCA and detection of the resulting RCPs with fluorescent probes.

### Example 2: Metal-Enhanced Fluorescence of Sequentially Generated Fluorescent Signals Corresponding to an Analyte in a Biological Sample Embedded in a 3D Hydrogel Matrix Attached to Metal Nanoparticles

This Example provides an example of a workflow for analyzing a biological sample embedded in a hydrogel matrix attached to metal nanoparticles which enhance fluorescent signals corresponding to analytes in situ in the biological sample.

A biological sample (e.g. a tissue section, cell pellet, cell block, or other sample) is embedded in a hydrogel comprising a matrix that is attached to metal nanoparticles. In parallel, a control biological sample is embedded in a hydrogel not comprising the metal nanoparticles.

In particular examples, the metal nanoparticles are gold nanoparticles, but in other examples metal nanoparticles comprise other and/or additional metals, such as any suitable metal provided herein. In some examples, the metal nanoparticles have an average diameter of between about 5 and about 250 nanometers (nm). In some examples, the metal nanoparticles are distributed throughout the 3D volume of the hydrogel.

The hydrogel comprising a matrix attached to metal nanoparticles can be provided using any suitable method. In this example, the metal nanoparticles are amine-functionalized gold nanoparticles, for example as illustrated in FIG. 3. The gold nanoparticles are reacted with a moiety containing an N-Hydroxysuccinimide (NHS) group and acrylic group, thereby forming gold nanoparticles covalently attached to acrylic groups. In some embodiments, Amine-functionalized gold nanoparticles are reacted with a moiety comprising an NHS ester moiety and acrylic group, thereby forming gold nanoparticles covalently attached to acrylic groups. The resulting acrylic-functionalized gold nanoparticles can then be polymerized alone or with other matrix-forming monomers to form the matrix of a hydrogel embedding the biological sample.

The biological sample (and control biological sample) is contacted with a circularizable probe (e.g. padlock probe) that hybridizes to a target nucleic acid, and the circularizable probe is ligated (and thereby circularized) using the target nucleic acid as template. The circularized probe is used as a template for RCA. An RCA primer is hybridized to the circularized probe, and an RCA reaction mixture (containing Phi29 reaction buffer, dNTPs, and Phi29 polymerase) is added to the sample. The sample is incubated at 37° C. for 3 hours to allow the RCA to proceed, thereby generating an RCP corresponding to the target nucleic acid, and then the RCA is terminated.

The sample is contacted with a first fluorescently labeled probe that hybridizes to the RCP, and the sample is imaged by fluorescence microscopy to detect fluorescent signals from the first fluorescently labeled probe at different 3D locations in the biological sample, which correspond to the target analyte. The sample is washed to remove the first fluorescently labeled probe. The sample is contacted with a second fluorescently labeled probe that hybridizes to the RCP, and the sample is imaged by fluorescence microscopy to detect fluorescent signals from the second fluorescently labeled probe at different 3D locations in the biological sample, which correspond to the target analyte.

The signals detected in the biological sample embedded in the hydrogel comprising the metal nanoparticles are compared to the signals detected in the control sample through multiple cycles of detection. For example, the fluorescent signals detected in the biological sample embedded in the hydrogel comprising the metal nanoparticles may have higher intensity, and may be detected at higher density.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
providing a biological sample embedded in a hydrogel matrix that is attached to metal nanoparticles;
generating a fluorescent signal associated with an analyte in the biological sample; and
detecting the fluorescent signal at a 3-dimensional (3D) location in the hydrogel matrix which corresponds to the 3D location of the analyte in the biological sample.

2. The method of claim 1, wherein the metal nanoparticles attached to the hydrogel matrix are distributed throughout the 3D volume of the hydrogel matrix.

3. The method of claim 1, wherein the metal nanoparticles enhance the fluorescent signal via metal-enhanced fluorescence (MEF).

4. (canceled)

5. The method of claim 1, wherein the fluorescent signal is generated from a fluorescent moiety that is between about 5 nm and about 90 nm from one or more of the metal nanoparticles attached to the hydrogel matrix.

6. The method of claim 1, wherein the analyte is a fluorescent analyte and the fluorescent signal is fluorescence of the analyte.

7. The method of claim 1, wherein the analyte is a nucleic acid analyte or a non-nucleic acid analyte.

8. The method of claim 1, wherein generating the fluorescent signal associated with the analyte comprises contacting the biological sample with a detectably labeled probe that binds directly or indirectly to the analyte or a product thereof, and using the detectably labeled probe to generate the fluorescent signal.

9-12. (canceled)

13. The method of claim 8, wherein generating the fluorescent signal associated with the analyte comprises performing rolling circle amplification (RCA) to generate an RCA product (RCP).

14. The method of claim 13, wherein the RCA is performed using as template a circular or circularized probe that binds directly or indirectly to the analyte or a product thereof, and wherein the detectably labeled probe binds directly or indirectly to the RCP.

15. (canceled)

16. The method of claim 8, wherein the analyte is a first analyte, the fluorescent signal is a first fluorescent signal, the detectably labeled probe is a first detectably labeled probe, and the method further comprises:
removing the first detectably labeled probe from the biological sample;
contacting the biological sample with a subsequent detectably labeled probe that binds directly or indirectly to a subsequent analyte or a product of the subsequent analyte in the biological sample and using the subsequent detectably labeled probe to generate a subsequent fluorescent signal; and
detecting the subsequent fluorescent signal at a 3D location in the hydrogel matrix that is attached to the metal nanoparticles, which corresponds to the 3D location of the subsequent analyte in the biological sample.

17-20. (canceled)

21. The method of claim 1, wherein the analyte is a first analyte, the fluorescent signal is a first fluorescent signal, and the first fluorescent signal is at a first 3D location; and wherein the method further comprises generating a second fluorescent signal associated with a second analyte in the biological sample, and detecting the second fluorescent signal at a second 3D location in the hydrogel matrix which corresponds to the 3D location of the second analyte in the biological sample, wherein the first and second 3D locations are different.

22-34. (canceled)

35. The method of claim 1, wherein the metal nanoparticles are attached to the hydrogel matrix via an attachment moiety that is linked to the metal nanoparticles.

36. The method of claim 35, wherein the attachment moiety is attached to an anchoring moiety in the hydrogel matrix, and wherein the attachment moiety and the anchoring moiety are a ligand-ligand binding pair or functional moieties that can react with each other.

37. The method of claim 1, wherein the hydrogel matrix is covalently attached to the metal nanoparticles.

38-43. (canceled)

44. The method of claim 1, wherein the metal nanoparticles comprise chromium, copper, gold, iron, nickel, platinum, silver, tin, zinc, or a combination thereof.

45. The method of claim 1, wherein the metal nanoparticles comprise gold.

46-50. (canceled)

51. The method of claim 1, wherein the concentration of metal nanoparticles attached to the hydrogel matrix is between about 10\hat{ }7 to 10\hat{ }15 metal nanoparticles per milliliter (mL).

52-56. (canceled)

57. The method of claim 1, wherein the metal nanoparticles comprise two or more different metal nanoparticle species, wherein two or more of the different metal nanoparticle species have different average diameters and/or comprise different metals or combinations thereof, and wherein the two or more different metal nanoparticle species enhance the intensity of fluorescent signals in different emission spectra.

58-68. (canceled)

69. A method, comprising:
(a) contacting a biological sample with a circular probe that hybridizes to an analyte or contacting the biological sample with a circularizable probe that hybridizes to the analyte and circularizing the circularizable probe to form a circularized probe;
(b) performing rolling circle amplification (RCA) using the circular or circularized probe as template to generate an RCA product (RCP);
wherein, prior to or after (a) and/or (b), the biological sample is embedded in a hydrogel matrix that is attached to metal nanoparticles;
(c) contacting the biological sample with a first detectably labeled probe that binds directly or indirectly to the RCP and detecting a fluorescent signal generated from the first detectably labeled probe at a 3-dimensional (3D) location in the hydrogel matrix which corresponds to the 3D location of the analyte in the biological sample;
(d) removing the first detectably labeled probe from the biological sample, optionally via one or more wash steps; and
(e) contacting the biological sample with a subsequent detectably labeled probe that binds directly or indirectly to the RCP and detecting a subsequent fluorescent signal generated from the subsequent detectably labeled probe at a 3-dimensional (3D) location in the hydrogel matrix which corresponds to the 3D location of the analyte in the biological sample.

70-71. (canceled)

72. A kit comprising:
a) a probe configured to bind directly or indirectly to an analyte in a biological sample, wherein the probe comprises a fluorescent label; and
b) i) hydrogel matrix forming monomers comprising metal nanoparticles, wherein the metal nanoparticles are capable of enhancing, via metal-enhanced fluorescence (MEF), a fluorescent signal generated from the fluorescent label; or
ii) hydrogel matrix forming monomers and metal nanoparticles configured to be attached to the hydrogel matrix forming monomers and/or to a hydrogel matrix formed from the hydrogel matrix forming monomers, wherein the metal nanoparticles are capable of enhancing, via metal-enhanced fluorescence (MEF), a fluorescent signal generated from the fluorescent label.

73-75. (canceled)
